(19) United States
(12) Patent Application Publication
(10) Pub. No.: US 2008/0234232 A1
(43) Pub. Date: Sep. 25, 2008

(54) Methods and compositions for targeting AFAP

(76) Inventors: Daniel C. Flynn, Morgantown, WV (US); Peter Gannett, Morgantown, WV (US)

Correspondence Address: BUCHANAN INGERSOLL & ROONEY PC, P.O. BOX 1404, ALEXANDRIA, VA, 22313-1404, US

(21) Appl. No.: 12/077,653

(22) Filed: Mar. 20, 2008

Related U.S. Application Data
(60) Provisional application No. 60/919,086, filed on Mar. 20, 2007.

(51) Int. Cl. A61K 31/661; A61P 35/00

(52) U.S. Cl. 514/121

(57) ABSTRACT

Embodiments of the invention are directed to methods and compositions for inhibiting activation of cSrc by human actin filament associated protein (AFAP). Libraries and methods of screening compositions for inhibitive activity are also provided. Also provided are methods of treating cancer. Cancer may be, for example, but is not limited to ovarian cancer, breast cancer, and gastrointestinal cancer. Also provided are methods of decreasing resistance to chemotherapy.

### CLAIM TO PRIORITY

This application claims priority to pending U.S. Provisional Patent Application No. 60/919,086, filed on Mar. 20, 2007, and incorporated by reference as if fully rewritten herein.

### STATEMENT REGARDING FEDERALLY SPONSORED OR DEVELOPMENT

This invention was made in the course of research sponsored by the National Institutes of Health. The U.S. Government may have certain rights in this invention.

### BACKGROUND OF THE INVENTION

1. Field of the Invention

Embodiments of the invention are directed to methods and compositions for inhibiting activation of cSrc by human actin filament associated protein (AFAP). Methods of screening compositions for such activity are also provided. Also provided are methods of treating cancer. Cancer may be, for example, but is not limited to ovarian cancer, breast cancer, and gastrointestinal cancer. Also provided are methods of decreasing resistance to chemotherapy.

2. Background of the Art

The cSrc (“Src”) nonreceptor tyrosine kinase is normally repressed and inactive in cells; however, during the G2/M transition, or responsive to growth factor receptor stimulation, Src becomes activated, concomitant with a relaxation of actin filament structures. Src is activated in several human cancer cell lines (Bolen et al., 1987, Proc. Natl. Acad. Sci. USA 84: 2251-2255; Boschek et al., 1981, Cell 24: 175-184; Cartwright et al., 1990, Proc. Natl. Acad. Sci. USA 87: 558-562; Irby et al. 1999, Nat. Genet. 21: 187-190; Rosen et al., 1986, J Biol. Chem. 261: 13754-13759; Tarone et al., 1985, Exp. Cell. Res. 159: 141-157) and one of the hallmarks of transformation by activated forms of Src is the dissolution of stress filaments and a repositioning of actin into rosette-like structures (Reynolds et al., 1989, Mol. Cell. Biol. 9: 3951-3958; Felice et al., 1990, Eur. J Cell Biol. 52: 47-59). Antisense vectors that reduce Src expression in the HT29 human colon cancer cell lines will significantly reduce the transformed properties of these lines and drugs that block Src will impede progression through the G2/M transition. These data demonstrate a role for Src in modulating signals that affect cell growth and motility.

The cSrc proto-oncogene can be activated by dephosphorylation of Tyr⁵²⁷ by cellular phosphatases, or displacement of repressive, intramolecular interactions involving the SH2 and SH3 domains (Brown and Cooper, 1996, Biochim. Biophys. Acta, 1287:121-149). These activation events normally occur in response to cellular signals, e.g., growth factors interacting with their receptors (Brown and Cooper, 1996, supra). These pathways are thought to proceed through Src, with the subsequent phosphorylation of substrates and activation of downstream signaling members, including Ras (He et al., 2000), pp125^{FAK} (Thomas et al., 1998, Exp. Cell Res., 159:141-157), Crk (Sabe et al., 1992, Mol. Cell Biol., 12: 4706-4713) and pp130^{Cas} (Xing et al., 2000, Mol. Cell Biol., 20: 7363-7377).

Downstream signaling proteins can modulate the effects of activated Src. For example, Src can be activated by dephosphorylation of Tyr⁵²⁷ by cellular phosphatases, or displacement of repressive, intramolecular interactions involving the SH2 and SH3 domains (Brown and Cooper, 1996, Biochem. Biophys. Acta 1287: 121-149). These activation events usually occur in response to cellular signals, e.g., such as occurs when growth factors interact with their receptors (Brown and Cooper, supra). Activated Src regulates actin filament integrity via signal transduction pathways modulated by downstream effector proteins, including PKCα, PI 3-kinase, Ras (He et al., 2000, Cancer J. 6: 243-248), pp125^{FAK} (Thomas et al., 1998, J. Biol. Chem. 273: 577-583) Crk (Sabe et al., 1992, supra), Rho and pp130^{Cas} (Xing et al., 2000, supra). Activated forms of PKCα, PI 3-kinase, and Ras can initiate changes in actin filaments similar to the effects of Src^{527F}. In addition, activation of Src will direct a down-regulation of Rho activity. While dominant negative forms of PKCα, PI 3-kinase, and Ras, will block the effects of Src^{527F} upon actin filaments, dominant-positive forms of Rho will direct the formation of well-formed stress fibers and block the ability of Src^{527F} to alter actin filament integrity.

The actin filament associated protein AFAP-110 is a tyrosine phosphorylated substrate of Src and is an SH2/SH3 binding partner for Src^{527F} (Flynn et al., 1993, Mol. Cell. Biol. 13: 7982-7900). AFAP-110 is an adaptor protein that binds to actin filaments via a carboxy terminal, actin binding domain and colocalizes with stress filaments and the cortical actin matrix along the cell membrane (Quin et al., 1998, Oncogene, 16: 2185-2195; Quin et al., 2000, Exp. Cell. Res., 255:1-2-113). AFAP-110 also is capable of being an SH2/SH3 binding partner for cFyn and cLyn (Flynn et al., 1993, supra; Guappone and Flynn, 1997, Mol. Carinogen. 22: 110-119). In addition to SH2 and SH3 binding motifs, AFAP comprises two pleckstrin homology domains (PH1 and PH2), a carboxy terminal leucine zipper, which facilitates self association of AFAP-110 (Quin et al., 1998, supra) and an actin binding domain (Flynn et al., supra, Qian et al, 2000, supra). AFAP-110 also contains a target region for serine/threonine phosphorylation as well as other hypothetical protein-binding sites (Baisden et al., 2001a, Oncogene, 20:6435-6447). AFAP-110 is hyperphosphorylated on ser/thr residues as well as tyrosine residues in Src transformed cells and contains numerous consensus sequences for phosphorylation by PKC (Kanner et al., 1991, EMBO J., 10:1689-1698; Flynn et al., 1993, supra). AFAP-110 appears to function as an adapter molecule linking a variety of signaling proteins to the actin cytoskeleton. This interaction is discussed more fully in United States Patent Application Publication No. US2003/0104443, incorporated by reference herein.

The Pleckstrin Homology (PH1) domain not only serves as a docking site for PKCα, but also plays a role in stabilizing AFAP-110 multimer formation (Qian, Y. et. al., 2002; Qian, Y. et. al., 2004). The PH1 domain of AFAP-110 contains a groove that is conserved among many PH domains and can serve as a binding pocket for phospholipids (Baisden, J. Met. al., 2001b). It has been reported that several PH domains can bind to phosphoinositots generated upon activation of phosphatidylinositol-3-kinase (PI3K) activity, such as Akt and DAPP I (Atessi, D. R. et. al., 1997)(Dowler, S. et. al., 1999).

The following documents, all of which are incorporated by reference herein, may be useful in understanding one or more embodiments of the present invention. Inclusion of a document anywhere in this specification is not an admission that document is prior art with respect to this application:
Alberti, S. (1998). A phosphoinositide-binding sequence is shared by PH domain target molecules--a model for the binding of PH domains to proteins. Proteins 31, 1-9.
Alessi, D. R., James, S. R., Downes, C. P., Holmes, A. B., Gaffney, P. R., Reese, C. B., and Cohen, P. (1997). Characterization of a 3-phosphoinositide-dependent protein kinase which phosphorylates and activates protein kinase Balpha. Curr. Biol. 7, 261-269.
Baisden, J. M., Gatesman, A. S., Cherezova, L., Jiang, B. H., and Flynn, D. C. (2001a). The intrinsic ability of AFAP-110 to alter actin filament integrity is linked with its ability to also activate cellular tyrosine kinases. Oncogene 20, 6607-6616.
Baisden, J. M., Qian, Y., Zot, H. M., and Flynn, D. C. (2001b). The actin filament-associated protein AFAP-110 is an adaptor protein that modulates changes in actin filament integrity. Oncogene 20, 6435-6447.
Balendran, A, Biondi, R. M., Cheung, P. C., Casamayor, A., Deak, M., and Alessi, D. R. (2000a). A 3-phosphoinositide-dependent protein kinase-1 (PDK1) docking site is required for the phosphorylation of protein kinase Czeta (PKCzeta) and PKC-related kinase 2 by PDKI. J. BioL Chem. 275, 20806-20813.
Balendran, A., Hare, G. R., Kieloch, A., Williams, M. R., and Alessi, D. R. (2000b). Further evidence that 3-phosphoinositide-dependent protein kinase-I (PDK1) is required for the stability and phosphoryiation of protein kinase C (PKC) isoforms. FEBS Lett. 484, 217-223.
Barry, O. P. and Kazanietz, M. G. (2001). Protein kinase C isozymes, novel phorbol ester receptors and cancer chemotherapy. Curr. Pharm. Des 7, 1725-1744.
Brachmann, S. M., Yballe, C. M., Innocenti, M., Deane, J. A., Fruman, D. A., Thomas, S. M., and Cantley, L. C. (2005). Role of phosphoinositide 3-kinase regulatory isoforms in development and actin rearrangement. Mol. Cell Biol. 25, 2593-2606.
Brandt, D., Gimona, M., Hillmann, M., Hailer, H., and Mischak, H. (2002). Protein kinase C induces actin reorganization via a Src- and Rho-dependent pathway. J. Biol. Chem. 277, 20903-20910.
Brandt, D. T., Goerke, A., Heuer, M., Gimona, M., Leitges, M., Kremmer, E., Lammers, R., Hailer, H., and Mischak, H. (2003). Protein kinase C delta induces Src kinase activity via activation of the protein tyrosine phosphatase PTP alpha. J. BioL Chem. 278, 34073-34078.
Bruce-Staskal, P. J. and Bouton, A. H. (2001). PKC-dependent activation of FAK and src induces tyrosine phosphorylation of Cas and formation of Cas-Crk complexes. Exp. Cell Res. 264, 296-306.
Burgering, B. M. and Coffer, P. J. (1995). Protein kinase B (c-Akt) in phosphatidylinositol-3-OH kinase signal transduction. Nature 376, 599-602.
Chen, R., Kang, V. H., Chen, J., Shope, J. C., Torabinejad, J., DeWald, D. B., and Prestwich, G. D. (2002). A monoclonal antibody to visualize Ptdlns(3,4,5)P(3) in ceils. J. Histochem. Cytochem. 50, 697-708.
Chun, Y. S., Lee, K. H., Choi, E., Bae, S. Y., Yeo, E. J., Huang, L. E., Kim, M. S., and Park, J. W. (2003). Phorbol ester stimulates the nonhypoxic induction of a novel hypoxia-inducible factor 1alpha isoform: implications for tumor promotion. Cancer Res. 63, 8700-8707.
Coghlan, M. P., Chou, M. M., and Carpenter, C. L. (2000). Atypical protein kinases Clambda and -zeta associate with the GTP-binding protein Cdc42 and mediate stress fiber loss. Mol. Cell Biol. 20, 2880-2889.
Cozier, G. E., Carlton, J., Bouyoucef, D., and Cullen, P. J. (2004). Membrane targeting by pleckstrin homology domains. Curt. Top. MicrobioL Immunol. 282, 49-88.
DeMatteis, M. A. and Godi, A. (2004). Protein-lipid interactions in membrane trafficking at the Golgi complex. Biochim. Biophys. Acta 1666, 264-274.
del Peso, L., Lucas, L., Esteve, P., and Lacal, J. C. (1997). Activation of phospholipase D by growth factors and oncogenes in murine fibroblasts follow alternative but cross-talking pathways. Biochem. J. 322 (Pt 2), 519-528.
Delage, S., Chastre, E., Empereur, S., Wicek, D, Veissiere, D., Capeau, J., Gespach, C., and Cherqui, G. (1993). Increased protein kinase C alpha expression in human colonic Caco-2 cells after insertion of human Ha-ras or polyoma virus middle T oncegenes. Cancer Res. 53, 2762-2770.

Dowler, S., Currie, R. A., Downes, C. P., and Alessi, D. R. (1999). DAPPI: a dual adaptor for phosphotyrosine and 3-phosphoinositides. Biochem. J. 342 (Pt 1), 7-12.
Downes, C. P., Gray, A., and Lucocq, J. M. (2005). Probing phospheinositide functions in signaling and membrane trafficking. Trends Cell Biol. 15, 259-268.
Dwyer-Nield, L. D., Miller, A. C., Neighbors, B. W., Dinsdale, D., and Malkinson, A. M. (1996). Cytoskeletal architecture in mouse lung epithelial ceils is regulated by protein-kinase C-alpha and calpain II. Am. J. Physio. 270, L526-L534.
Ferguson, K. M., Kavran, J. M., Sankaran, V. G., Fournier, E., Isakoff, S. J., Skolnik, E. Y., and Lemmon, M. A. (2000). Structural basis for discrimination of 3-phosphoinositides by pleckstrin homology domains. Mol. Cell 6,373-384.
Foster, D. A. and Xu, L. (2003). Phospholipase D in cell proliferation and cancer. Mol. Cancer Res. 1,789-800.
Frank, S. R., Hatfield, J. C., and Casanova, J. E. (1998). Remodeling of the actin cytoskeleton is coordinately regulated by protein kinase C and the ADP-ribosylation factor nucleotide exchange factor ARNO. Mol. Biol. Cell 9, 3133-3146.
Fruman, D. Ao, Mauvais-Jarvis, F., Pollard, D. A., Yballe, C. M., Brazil, D., Bronson, R. T., Kahn, C. R., and Cantley, L. C. (2000). Hypogtycaemia, liver necrosis and perinatal death in mice lacking all isoforms of phosphoinositide 3-kinase p85 alpha. Nat. Genet. 26, 379-382.
Fruman, D. A., Meyers, R. E., and Cantley, L. C. (1998). Phosphoinositide kinases. Annu. Rev. Biochem. 67, 481-507.
Gatesman, A., Walker, V. G., Baisden, J. M., Weed, S. A., and Flynn, D. C. (2004) Protein kinase Calpha activates c-Src and induces podosome formation via AFAP-110. Mol. Cell Biol 24, 7578-7597.
Gibson, T. J., Hyvonen, M., Musacchio, A., Saraste, M., and Birney, E. (1994). PH domain: the first anniversary. Trends Biochem. Sci. 19, 349-353.
Gliki, G., Wheeler-Jones, C., and Zachary, I. (2002) Vascular endothelial growth factor induces protein kinase C (PKC)-dependent Akt/PKB activation and phosphatidylinositol 3′-kinase-mediates PKC delta phosphorylation: role of PKC in angiogenesis. Cell Biol. Int. 26, 751-759.
Guappone, A. C. and Flynn, D. C. (1997) The integrity of the SH3 binding motif of AFAP-110 is required to facilitate tyrosine phosphorylation by, and stable complex formation with, Src. Mol. Cell Biochem. 175, 243-252.
Guappone, A. C., Qian, Y., Weimer, T., and Flynn, D. C. (1996). An in vivo system for analysis of stable complex formation between Src and AFAP-110. Methods in Cell Science 18, 55-65.
Hai, C. M., Hahne, P., Harrington, E. O., and Gimona, M. (2002) Conventional protein kinase C mediates phorbol-dibutyrate-induced cytoskeletal remodeling in a7r5 smooth muscle cells. Exp. Cell Res. 280, 64-74.
Hama, H., Torabinejad, J., Prestwich, G. D., and DeWald, D. B. (2004). Measurement and immunofluorescence of cellular phosphoinositides. Methods Mol. Biol. 284, 243-258.
Harrington, E. O., Loftier, J., Nelson, P. R., Kent, K. Co, Simons, M., and Ware, J. A. (1997). Enhancement of migration by protein kinase Calpha and inhibition of proliferation and cell cycle progression by protein kinase Cdelta in capillary endothelial ceils. J. Biol. Chem. 272, 7390-7397.
Helms, J. B. and Zurzolo, C. (2004). Lipids as targeting signals: lipid rafts and intracellular trafficking. Traffic. 5, 247-254.
Hyvonen, M., Macias, M. J., Nilges, M., Oschkinat, H., Saraste, M., and Wilmanns, M. (1995). Structure of the binding site for inositol phosphates in a PH domain. EMBO J. 14, 4676-4685.
Imamura, H., Takaishi, K., Nakano, K., Kodama, A., Oishi, H., Shiozaki, H., Monden, M., Sasaki, T., and Takai, Y. (1998). Rho and Rab small G proteins coordinately reorganize stress fibers and focal adhesions in MDCK cells. Mol. Biol. Cell 9, 2561-2575.
Isakoff, S. J., Cardozo, T., Andreev, J., Li, Z., Ferguson, K. M., Abagyan, R., Lemmon, M. A., Aronheim, A., and Skolnik, E. Y. (1998) Identification and analysis of PH domain-containing targets of phosphatidylinositol 3-kinase using a novel in vivo assay in yeast. EMBO J. 17, 5374-5387.
Jaken, S., Leach, K., and Klauck, T. (1989). Association of type 3 protein kinase C with focal contacts in rat embryo fibroblasts. J Cell Biol. 109, 697-704.
Jiang, B. H., Aoki, M., Zheng, J. Z., Li, Jo, and Vogt, P. K. (1999). Myogenic signaling of phosphatidylinositol 3-kinase requires the serine-threonine kinase Akt/protein kinase B. Proc. Natl. Acad. Sci. U.S.A. 96, 2077-2081.
Jiang, B. H., Zheng, J. Z., and Vogt, P. K. (1998). An essential role of phosphatidylinositol 3-kinase in myogenic differentiation. Proc. Natl. Acad. Sci. U.S.A. 95, 14179-14183.
Kaplan, K. B., Swedlow, J. R., Varmus, H. E., and Morgan, D. O. (1992) Association of p60c-src with endosomal membranes in mammalian fibroblasts. J. Cell Biol. 118, 321-333.
Katso, R., Okkenhaug, K., Ahmadi, K., White, S., Timms, J., and Waterfield, M. D. (2001). Cellular function of phosphoinositide 3-kinases: implications for development, homeostasis, and cancer. Annu. Rev. Cell Dev. Biol. 17, 615-675.
Kawakami, Y., Nishimoto, H., Kitaura, J., Maeda-Yamamoto, M., Kato, R. M., Littman, D. R., Rawlings, D. J., and Kawakami, T. (2004) Protein kinase C betall regulates Akt phosphorylation on Ser-473 in a cell type- and stimulus-specific fashion. J. Biol. Chem. 279, 47720-47725.
Kazanietz, M. G. (2000). Eyes wide shut: protein kinase C isozymes are not the only receptors for the phorbol ester tumor promoters. Mol. Carcinog. 28, 5-11.
Kazanietz, M. G., Caloca, M. J., Eroles, P., Fujii, T., Garcia-Bermejo, M. L., Reilly, M., and Wang, H. (2000). Pharmacology of the receptors for the phorbol ester tumor promoters: multiple receptors with different biochemical properties. Biochem. Pharmacol. 60, 1417-1424.
Kiley, S. C., Parker, P. J., Fabbro, D., and Jaken, S. (1992) .Hormone- and phorbol ester-activated protein kinase C isozymes mediate a reorganization of the actin cytoskeleton associated with prolactin secretion in GH4C1 cells. Mol. Endocrinol. 6, 120-131.
Klinghoffer, R. A., Sachsenmaier, C., Cooper, J. A., and Soriano, P. (1999). Src family kinases are required for integrin but not PDGFR signal transduction. EMBO J. 18, 2459-2471.
Klippel, A., Kavanaugh, W. M., Pot, D., and Williams, L. T. (1997). A specific product of phosphatidylinositol 3-kinase directly activates the protein kinase Akt through its pleckstrin homology domain. Mol. Cell Biol. 17, 338-344.
Kolanus, W. and Seed, B. (1997). Integrins and inside-out signal transduction: converging signals from PKC and PIP3. Curr. Opin. Cell Biol. 9, 725-731.
Lemmon, M. A. (2003). Phosphoinositide recognition domains. Traffic. 4, 201-213.
Lemmon, M. A. (2004). Pleckstrin homology domains: not just for phosphoinositides. Biochem. Soc. Trans. 32, 707-711.
Lemmon, M. A. (2005). Pleckstrin homology domains: two halves make a hole? Cell 120, 574-576.
Lemmon, M. A. and Ferguson, K. M. (2000). Signal-dependent membrane targeting by pleckstrin homology (PH) domains. Biochem. J. 350 Pt 1, 1-18.
Liscovitch, M., Czarny, M., Fiucci, G., Lavie, Y., and Tang, X. (1999). Localization and possible functions of phospholipase D isozymes. Biochim. Biophys. Acta 1439, 245-263.
Macias, M. J., Musacchio, A., Ponstingl, H., Nilges, M., Saraste, M., and Oschkinat, H. (1994). Structure of the pleckstrin homology domain from beta-spectrin. Nature 369, 675-677.
Musacchio, A., Gibson, T., Rice, P., Thompson, J., and Saraste, M. (1993). The PH domain: a common piece in the structural patchwork of signalling proteins. Trends Biochem. Sci. 18, 343-348.
O'Luanaigh, N., Pardo, R., Fensome, A., Allen-Baume, V., Jones, D., Holt, M. R., and Cockcroft, S. (2002). Continual production of phosphatidic acid by phospholipase D is essential for antigen-stimulated membrane ruffling in cultured mast cells. Mol. Biol. Cell 13, 3730-3746.
Obenauer, J. C., Cantley, L. C., and Yaffe, M. B. (2003). Scansite 2.0: Proteome-wide prediction of cell signaling interactions using short sequence motifs. Nucleic Acids Res. 31, 3635-3641.
Petersen, F. N., Jensen, M. O., and Nielsen, C. H. (2005) Interfacial tryptophan residues: a role for the cation-pi effect? Biophys. J. 89, 3985-3996.
Qian, Y., Baisden, J. M., Cherezova, L., Summy, J. M., Guappone-Koay, A., Shi, X., Mast, T., Pustula, J., Zot, H. G., Mazloum, N., Lee, M. Y., and Flynn, D. C. (2002). PC phosphorylation increases the ability of AFAP-110 to cross-link actin filaments. Mol. Biol. Cell 13, 2311-2322.
Qian, Y., Baisden, J. M., Westin, E. H., Guappone, A. C., Koay, T. C., and Flynn, D. C. (1998). Src can regulate carboxy terminal interactions with AFAP-110, which influence self-association, cell localization and actin filament integrity. Oncogene 16, 2185-2195.
Qian, Y., Baisden, J. M., Zot, H. G., Van Winkle, W. B., and Flynn, D. C. (2000). The carboxy terminus of AFAP-110 modulates direct interactions with actin filaments and regulates its ability to alter actin filament integrity and induce lamellipodia formation. Exp. Cell Res. 255, 102-113.
Qian, Y., Gatesman, A. S., Baisden, J. Mo, Zot, H. G., Cherezova, L., Qazi, I., Mazloum, N., Lee, M. Y., Guappone-Koay, A., and Flynn, D. C. (2004). Analysis of the role of the leucine zipper motif in regulating the ability of AFAP-110 to alter actin filament integrity. J. Ceil Biochem. 91,602-620.
Qian, Y., Guappone, A. C., Baisden, J. M., Hill, M. W., Summy, J. M., and Flynn, D. C. (1999) Monoclonal antibodies directed against AFAP-110 recognize species-specific and conserved epitopes. Hybridoma 18, 167-175.
Qureshi, S. A., Joseph, C. K., Rim, M., Maroney, A., and Foster, D. A. (1991). v-Src activates both protein kinase C-dependent and independent signaling pathways in murine fibroblasts. Oncogene 6, 995-999.
Redmond, T., Brott, B. K., Jove, R., and Welsh, M. J. (1992). Localization of the viral and cellular Src kinases to perinuclear vesicles in fibroblasts. Cell Growth Differ. 3, 567-576.
Reynolds, A. B., Kanner, S. B., Wang, H. C., and Parsons, J. T. (1989). Stable association of activated pp60src with two tyrosine-phosphorylated cellular proteins. Mol. Cell Biol. 9, 3951-3958.
Rizzo, M. and Romero, G. (2002). Pharmacological importance of phospholipase D and phosphatidic acid in the regulation of the mitogen-activated protein kinase cascade. Pharmacol. Ther. 94, 35-50.
Roth, M. G., Bi, K., Ktistakis, N. T., and Yu, S. (1999). Phospholipase D as an effector for ADP-ribosylation factor in the regulation of vesicular traffic. Chem. Phys. Lipids 98, 141-152.
Roth, M. G. and Sternweis, P. C. (1997). The role of lipid signaling in constitutive membrane traffic. Curr. Opin. Cell Biol. 9, 519-526.
Salaun, C., James, D. J., and Chamberlain, L. H. (2004). Lipid rafts and the regulation of exocytesis. Traffic. 5, 255-264.
Sandilands, E., Cans, C., Fincham, Vo J., Brunton, V. G., Mellor, H., Prendergast, G. C., Norman, J. C., Superti-Furga, G., and Frame, M. C. (2004). RhoB and actin polymerization coordinate Src activation with endosome-mediated delivery to the membrane. Dev. Cell 7, 855-869.
Shaw, G. (1993). Identification of novel pleckstrin homology (PH) domains provides a hypothesis for PH domain function. Biochem. Biophys. Res. Commun. 195, 1145-1151.
Siddiqi, A. R., Srajer, G. E., and Leslie, C. C. (2000) Regulation of human PLD1 and PLD2 by calcium and protein kinase C. Biochim. Biophys. Acta 1497, 103-114.
Stevenson, J. M., Perera, I. Y., and Boss, W. F. (1998). A phosphatidylinositol 4-kinase pleckstrin homology domain that binds phosphatidylinositol 4-monophosphate. J. Biol. Chem. 273, 22761-22767.
Teti, A., Colucci, S., Grano, M., Argentine, L., and Zambonin, Z. A. (1992). Protein kinase C affects microfilaments, bone resorption, and [Ca2+]o sensing in cultured osteoclasts. Am. J. Physiol 263, C130-C139.
Thomas, C. C., Deak, M., Alessi, D. R., and van Aalten, D. M. (2002). High-resolution structure of the pleckstrin homology domain of protein kinase b/akt bound to phosphatidylinositol (3,4,5)-trisphosphate. Curr. Biol. 12, 1256-1262.
Thomas, C. C., Dowler, S., Deak, M., Alessi, D. R., and van Aalten, D. M. (2001). Crystal structure of the phosphatidylinositol 3,4-bisphosphate-binding pleckstrin homology (PH) domain of tandem PH-domain-containing protein 1 (TAPP1): molecular basis of lipid specificity. Biochem. J. 358, 287-294.
Ueki, K., Yballe, C. M., Brachmann, S. M., Vicent, D., Watt, J. M., Kahn, C. R., and Cantley, L. C. (2002). Increased insulin sensitivity in mice lacking p85beta subunit of phosphoinositide 3-kinase. Proc. Natl. Acad. Sci. U.S.A. 99, 419-424.
Wang, Q., Bilan, P. J., Tsakiridis, T., Hinek, A., and Klip, A. (1998). Actin filaments participate in the relocalization of phosphatidylinositol3-kinase to glucose transporter-containing compartments and in the stimulation of glucose uptake in 3T3-L I adipocytes. Biochem. J. 331 (Pt 3), 917-928.
Wymann, M. P. and Marone, R. (2005). Phosphoinositide 3-kinase in disease: timing, location, and scaffolding. Curr. Opin. Cell Biol. 17, 141-149.
Wymann, M. P., Zvelebil, M., and Laffargue, M. (2003). Phosphoinositide 3-kinase signalling—which way to target? Trends Pharmacol. Sci. 24, 366-376.
Xu, Lo, Shen, Yo, Joseph, T., Bryant, A., Luo, J. Q., Frankel, P., Rotunda, T., and Foster, D. A. (2000). Mitogenic phospholipase D activity is restricted to caveolin-enriched membrane microdomains. Biochem. Biophys. Res. Commun. 273, 77-83.
Yeatman, T. J. (2004). A renaissance for SRC. Nat. Rev. Cancer 4, 470-480.
Yeo, E. J., Provost, J. J., and Extort, J. H. (1997). Dissociation of tyrosine phosphorylation and activation of phosphoinositide phospholipase C induced by the protein kinase C inhibitor Ro-31-8220 in Swiss 3T3 cells treated with platetet-derived growth factor. Biochim. Biophys. Acta 1356, 308-320.
Yoon, H. S., Hajduk, P. J., Petros, A. M., Olejniczak, E. T., Meadows, R. P., and Fesik, S. W. (1994). Solution structure of a pleckstrin-homology domain. Nature 369, 672-675.
Yu, J., Zhang, Y., Mcllroy, J., Rordotf-Nikolic, T., Orr, G. A., and Backer, J. M. (1998). Regulation of the p85/p110 phosphatidylinositol 3′-kinase: stabilization and inhibition of the p110alpha catalytic subunit by the p85 regulatory subunit. Mol. Cell Biol. 18, 1379-1387.
Zang, Q., Frankel, P., and Foster, D. A. (1995). Selective activation of protein kinase C isoforms by v-Src. Cell Growth Differ. 6, 1367-1373.
Zheng, Y. (2001). Dbl family guanine nucleotide exchange factors. Trends Biochem. Sci. 26, 724-732.
Zheng, Y., Zangrilli, D., Cerione, R. A., and Eva, A. (1996). The pleckstrin homology domain mediates transformation by oncogenic dbl through specific intracellular targeting. J. Biol. Chem. 271, 19017-19020.

### BRIEF SUMMARY OF THE INVENTION

PMA-directed activation of PKCa will induce the cSrc binding partner AFAP-110 to colocalize with and activate cSrc. The ability of AFAP-110 to colocalize with cSrc is dependent upon the integrity of the amino terminal Pleckstrin Homology (PH1) domain, while the ability to activate cSrc is dependent upon the integrity of its SH3 binding motif, which engages the cSrc SH3 domain. The outcome of AFAP-110-directed cSrc activation is a change in actin filament integrity and the formation of ventral membrane structures that resemble podosomes or precursors to invadopodia.

Embodiments of the invention address altering the ability of AFAP-110 to colocalize with cSrc in response to PMA. Treatment of mouse embryo fibroblast with a PI3K inhibitor, LY294002, blocks PMA-directed colocalization between AFAP-110 and cSrc and subsequent cSrc activation. PMA was unable to induce colocalization or cSrc activation in cells that lacked the p85α and β regulatory subunits of PI3K. In normal mouse embryo fibroblasts, PMA was able to induce activation of PI3K and the PH1 domain of AFAP-110 was capable of binding to phosphoinositide lipids, in vitro. These data indicate that PI3K activity is required for PMA-induced colocalization between AFAP-110 and cSrc and subsequent cSrc activation.

Embodiments of the invention provide methods that may be used to treat diseases where cSrc is activate, or as a preventative drug that can block cSrc activation. This may prevent or slow the progression of cancer. For example, cancer may be, but is not limited to breast, ovarian, brain, or colon cancer. Embodiments of the invention also provide methods and compositions for blocking cSrc family kinase activation associated with allergies. For example, it may be used to block activation of Lyn.

Embodiments of the invention also provide compositions and methods of use of compositions that block cSrc activation. These compositions may be, for example, phosphatidic acid or derivatives of phosphatidic acid. Compositions of the invention may target the amino terminal PH domain of AFAP-110.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1: LY294002 blocks PMA-induced colocalization between AFAP-110 and cSrc, and subsequent cSrc activation. A) SYF cells transiently co-expressing GFP-AFAP-110 and cSrc were untreated (panels a-d), treated with 100 nM PMA for 15 minutes (panels e-h), or treated with PMA and either 6 μM bisindolylmaleimide (panels i-I), 10 μM LY294002 (panels m-p) or 50 nM Wortmannin (panels q-t). Fixed cells were immunolabeled with avian specific cSrc antibody, EC10 (1:500), and phospho-Src-family (Tyr416) (1:250) as described in the methods section. Secondary antibodies used were Atexa 546 anti-mouse for EC10 and Alexa 647 anti-rabbit for phospho-Src-family (Tyr416). A merged image was generated to determine colocalization between AFAP-110 and cSrc. Bars represent 20 μM.

B) Western blot analysis using 40 μg of protein was performed as described in the method section of the examples below. Knockout cells (p85−/−) were compared to system control cells SYF and SYF/cSrc. Membranes were probed with antibodies to AFAP-110, p85α, PKCα and cSrc. Membranes were stripped and re-probed with anti-β-actin as a loading control.

C) Western blot analysis using 40 μg of protein was performed as described in the method section. Knockout cells (p85−/−) were compared to system control cells SYF and SYF/cSrc. Membranes were probed with an antibody that recognizes p110α and re-probed with anti β-actin.

D) Knockout MEFs (p85−/−) were transfected with GFP-AFAP-110 and cSrc. Fixed cells were immunolabeled with EC10 (1:500) and phospho-Src-family (Tyr416) (1:250) as described in the methods section. Secondary antibodies used were Alexa 546 anti-mouse for EC10 and Alexa 647 anti-rabbit for phospho-Src-family (Tyr416). Cells treated with 100 riM PMA for 15 minutes (panels e-h). Images were merged to determine colocalization of cSrc and GFP-AFAP-110. Bars represent 20 μm. Mouse embryo fibroblast cells that express the p85α isoform of the PI3K regulatory subunit (p85α+/+) were transfected with GFP-AFAP-110 and cSrc. Fixed cells were immunolabeled with EC10 (1:500) and phospho-Src-family (Tyr416) (1:250) as described in the methods section. Secondary antibodies used were Alexa 546 anti-mouse for EC10 and Alexa 647 anti-rabbit for phospho-Src-family (Tyr416). Images were merged to determine colocalization of cSrc and GFP-AFAP-110. Bars represent 20 μm.

E) SYF, SYF/cSrc, and p85−/− were treated with 100 nM PMA for 15 minutes, 40 μg cell lysate resolved by SDS-PAGE and western blot analysis performed to evaluate phospho-Src family (Y416)(1:1000) and cSrc (1:500) as described in the methods section. Beta-actin (1:5000) was used as a loading control.

F) SYF/cSrc cells transfected with GFP-AFAP-110 with or without myrPKCα. The cells were and immunolabeled with Flag antibody (1:1000), and phospho-Src-family (Tyr416) (1:250) as described in the methods section. Secondary antibodies used were Alexa 546 anti-mouse for Flag and Alexa 647 anti-rabbit for phospho-Src (Tyr416). A merged image used to determine colocalization between AFAP-110 and myrPKα. Controls without myrPKCα untreated (panels a-d) or treated with 20 μM LY294002 (panels e-h). Cells co-expressing GFP-AFAP-110 and myrPKCα show an increase in cSrc phosphorylation (panels i-I) and colocalization between myrPKCα and AFAP-110 (panels i-I). Treatment of cells treated with 20 μM LY abrogated colocalization and cSrc phosphorylation (panels m-p).

G) Mouse embryo fibroblast p85−/− cells expressing myrPKCα were and immunolabeled with Flag antibody (1:000), TRITC-phailoidin (1:500) and phospho-Src-family (Tyr416) (1:250) as described in the methods section. Secondary antibody used was Alexa 488 anti-mouse for Flag. A merged image was generated to determine colocalization between actin and myrPKCα. The myrPKCα failed to induce an increase in cSrc activation (panels e-h) or disruption of the actin cytoskeleton (panel e-h). Bars represent 20 μM.

FIG. 2: Inhibition of PI3K blocks translocation of cSrc from the perinuclear region of mouse embryo fibroblast in response to PMA treatment. SYF and p85−/− null cells were transiently transfected with cSrc and labeled for avian cSrc using EC10, an avian-specific monoclonal antibody (1:500). EC10 was visualized with anti-mouse Atexa Fluor 488 (false colored red for consistency). Cells treated with 100 nM PMA (panel b) induced translocation of cSrc to the cell periphery as indicated by the arrows. However, cells treated with 100 nM PMA in conjunction with 6 μM bisindolylmaleimide (panel c) or 10 μM LY (panel d) blocked the translocation of cSrc as indicated by the arrows. In p85−/− null cells that were treated with 100 nM PMA (panel f) failed to induce the translocation of cSrc to the cell periphery, as indicated by the arrows. (n=nucleus) Bars represent 20 μM.

FIG. 3: PMA treatment results in an increase in PI3K activation, in vitro. SYF/cSrc cells were cultured under serum-free conditions for 24 hours. Cells were unstimulated or stimulated with 10% serum (positive control), 100 nM PMA for 5 or 15 minutes. Cells were then lysed in kinase assay buffer and a PI3K kinase activity assay performed using anti-p1100α immunoprecipitates to measure PMA-induced incorporation of [³²p] into phosphoinositide substrates.

FIG. 4: PMA treatment results in an increase in PI3K activity, in vivo. SYF/cSrc cells were serum-starved for 24 hours. Cells stimulated with 10% serum (panels c-f), 100 nM PMA for 5 (panels g-h), or 15 minutes (panels i-j); as well as treated with 100 nM PMA in combination with 6 μM bisindolylmaleimide [I] (panels k-I) or 50 nM wortmannin (panels m-n). Cells treated with 100 nM PMA in combination with 5 μM PP1 (panel o-p) and Ptdlns-3,4,5-P₃ levels evaluated. Cells were immunolabeled with a PI-3,4,5-P₃-specific antibody (Echelon, Inc.;1:100) and TRITC-phalloidin (1:500). Secondary antibody for anti-PI-3,4,5-P₃ (1:100) was Alexa IgM 647 anti-mouse (1:200). Bars represent 20 μm.

FIG. 5: The phospholipid binding properties of the PH domains of AFAP-110. A) Pleckstrin homology fusion proteins, GST-PH1, GST-PH2 and GST-DAPP1, were purified from bacteria by affinity chromatography. Phospholipids were spotted onto PVDF membranes as described in the methods section. The membranes were incubated overnight with 0.5 μg/ml
GST-PH1, GST-PH2 or GST-DAPP1. Washed membranes were incubated with anti-GST antibody and exposed to film. Developed films showed the location and intensities of bound fusion proteins.

B) Pleckstrin homology fusion protein, GST-PH1, was purified from bacteria by affinity chromatography. Two-fold changes in concentration (1.6 pg-100.0 pg) of phospholipids were spotted onto PVDF membranes as described in the methods section. The PIP arrays were incubated with 0.5 μg/ml fusion protein and probed with anti-GST antibody.

C) Fusion protein, GST-DAPP1, was purified from bacteria by affinity chromatography. Two-fold changes in concentration (1.6 pg-100.0 pg) of phospholipids were spotted onto PVDF membranes as described in the methods section. Membranes were incubated with 0.5 μg/ml fusion proteins. Washed membranes were probed using with anti-GST antibody.

D) Full-length AFAP-110 GST fusion protein was purified from bacteria by affinity chromatography. Two-fold changes in concentration (1.6 pg-100.0 pg) of phospholipids were spotted onto PVDF membranes as described in the methods section. Membranes were incubated with 2.0 μg/ml fusion proteins. Membranes were probed using anti-GST antibody.

E) Lipid sedimentation assay was performed using pleckstrin homology fusion protein GST-PH1 as described in the methods sections. The GST-PH1 fusion protein has the capacity to co-sediment with several lipids containing vesicles as indicated in the pellet (P). Soluble fractions (S) containing unbound GST-PH1 purified fusion proteins were resolved by SDS-PAGE and detected by Coomassie stain.

FIG. 6: Molecular modeling of the PH1 domain reveals a mechanism to bind phosphoinositide-containing lipids. Computer generated model comparing (A) the binding pocket of a known PH domain (Macias, M. J. et. al., 1994) and (B) loop 7 of the PH 1 domain of AFAP-110. The positions of the conserved Arginine, Lysine, and Tryptophan residues are indicated and their relationship in the binding pocket.

FIG. 7: AFAP-110 PH1 integrity is required for cSrc colocalization and activation in response to PMA treatment. A) SYF cells transiently co-expressing GFP-AFAP-110 (panels a-h) or GFP-AFAP-110^{W169A} (panels i-p) and cSrc were untreated or pretreated with PMA and immunolabeled with avian specific cSrc antibody, EC10 (1:500), and phospho-Src-family (Tyr416) (1:250) as described in the methods section. Secondary antibodies used were Alexa 546 anti-mouse for EC10 and Alexa 647 anti-rabbit for phospho-Src-family (Tyr416). Bars represent 20 μm. B) SYF cells expressing GFP-AFAP-110^{Δ180-226} (panels a-h) or co-expressing GFP-AFAP-110 and Flag-AFAP-110^{Δ180-226} (panels i-p) then left untreated or pretreated with 100 nM PMA, fixed and immunolabeled with phospho-Src-family (Tyr416) (1:250) and anti-flag (1:1000) as described in the methods section. Secondary antibodies used, were Alexa 546 anti-mouse for flag and Alexa 647 anti-rabbit for phospho-Src-family (Tyr416). Bars represent 20 μm.

FIG. 8: Structure of lipid products screened for AFAP-110 PH1 domain binding capacities. Schematic structure representation of phosphotipids and other lipid products that were tested in this manuscript. The number in the bracket represents a qualitative approximation of the binding efficiency of each lipid for the PH1 domain as shown in FIG. 5A.

### DETAILED DESCRIPTION OF THE INVENTION

It has been reported that PMA or myrPKCα could direct activation of cSrc in a fashion dependent upon AFAP-110 (Gatesman, A. et. al., 2004). There is significant evidence to indicate the existence of cross talk between PKCα, cSrc and phosphatidylinositol-3-kinase (PI3K). Activation of each of these kinases results in substantial changes in actin filament integrity and cell morphology concomitant with increased cell motility and invasive potential (Coghlan, M. P. et. al., 2000; Dwyer-Nield, L. D. et. al., 1996; Frank, S. R. et. al., 1998; Harrington, E. O. et. al., 1997; Imamura, H. et. al., 1998; Jaken, S. et. al., 1989; Kiley, S. C. et. al., 1992; Teti, A. et. al., 1992) (reviewed in (Yeatman, T. J., 2004)). The ability of cSrc and PKCα to cross talk was determined by the use of a constitutively active form of cSrc or stable expression of viral-Src (v-Src), which will stimulate an increase in PKCα signaling, indicating that PKCα could function downstream of cSrc (Delage, S. et. al., 1993; Qureshi, S. A. et. al., 1991; Zang, Q. et. al., 1995). Alternatively, other studies have demonstrated that PKCα can function upstream and direct activation of cSrc (Brandt, D. et. al., 2002; Brandt, D. T. et. al., 2003; Bruce-Staskal, P. J. et. al., 2001).

Although PKCα can phosphorylate cSrc, in vitro studies have demonstrated that PKCα does not activate cSrc directly (Brandt, D. T. et. at., 2003). It has been reported that the PKCα and cSrc binding partner, actin filament-associated protein (AFAP-110), is able to relay signals from PKCα that direct activation of cSrc (Gatesman, A. et. al., 2004). We have found that expression of myristylated PKCα (myrPKCα) or treatments of cells with phorbol 12-myristate 13-acetate (PMA) induced AFAP-110 to colocalize with cSrc and subsequently activate it. The ability of AFAP-110 to colocalize with cSrc was dependent upon the integrity of the amino terminal pleckstrin homology (PH1) domain, while the ability of AFAP-110 to activate cSrc was dependent upon the integrity of the proline-rich SH3 binding motif in AFAP-110, which contacts the SH3 domain of cSrc. Thus, AFAP-110 is able to integrate signals from PMA or myrPKCα that enable it to colocalize with and subsequently activate cSrc. The integrity of the PH1 domain appears essential for AFAP-110 to colocalize with cSrc. PH domains are self-folding modular domains that are known to bind both proteins and lipids (Lemmon, M. A., 2004). Recent data indicate that many PH domains are able to bind to lipid products generated by PI3K (Cozier, G. E. et. al., 2004; De Matteis, M. A. et. al., 2004; Dowries, C. P. et. al., 2005; Helms, J. B. et. al., 2004; Lemmon, M. A., 2004; Lemmon, M. A., 2005; Salaun, C. et. al., 2004). An example is Akt, which upon activation by PI3K, will bind to phosphoinositol-3,4,5-trisphosphate (Ptdlns-3,4,5-P₃) via its PH domain, enabling Akt to traffic to the cell membrane, where it becomes phosphorylated by PDK-1 and activated (Alessi, D. R. et. al., 1997). It has been reported that many PH domains contain a binding groove that allows them to coordinate lipid binding. Thus, it was proposed that lipid binding to PH domains might promote translocation by facilitating interactions with membranes.

As colocalization and subsequent cSrc activation was dependent upon the integrity of the PHI domain, and molecular modeling analysis indicated that AFAP-110's PH1 domain had the capacity to bind lipids (Baisden, J. M. et. al., 2001b), applicants considered that PI3K may play a rote in facilitating colocalization between AFAP-110 and cSrc in response to PMA. Applicants recognize that (a) in cells where PI3K activity is blocked or PI3K regulatory subunits are deleted, PMA would fail to direct AFAP-110 to colocalize with cSrc; (b) loss of PI3K protein expression or activity would prevent PMA-induced activation of cSrc; (c) PMA should be able to direct activation of PI3K, possibly in a PKCα-dependent manner and (d) AFAP-110 should have the capacity to bind phosphoinositides via its PH1 domain.

Applicants have demonstrated that PMA or myrPKCα was directing AFAP-110 to colocalize with and subsequently activate cSrc. Applicants have determined that the ability of AFAP-110 to colocalize with cSrc is dependent upon the integrity of its PH1 domain. The PH1 domain not only serves as a docking site for PKCα, but also plays a role in stabilizing AFAP-110 multimer formation (Qian, Y. et. al., 2002; Qian, Y. et. al., 2004). It was hypothesized that PKCα binding to the PH1 domain could displace intramolecular interactions that autoinhibit AFAP-110 from moving to and activated cSrc (Qian, Y. et. al., 2002; Qian, Y. et. al, 2004). Without wishing to be bound by theory, applicants considered that activation of PKCα resulted in phosphorylation of AFAP-110, which affected a conformational change that displaced the leucine zipper motif from binding to the PH1 domain, effectively releasing autoinhibition and enabling AFAP-110 to move to and activate cSrc. These data were supported by the observation that phosphorylation of AFAP-110 in vitro (using a 20:1 ratio of recombinant AFAP-110 to recombinant PKCα) destabilized the AFAP-110 multimer (Qian, Y. et. al, 2004). Interestingly, deletion of amino acids 180-226 in the PH1 domain also destabilized the multimer but was not sufficient to direct AFAP-110 to colocalize or activate cSrc. In fact, deletion of amino acids 180-226 prevented PKCα from directing AFAP^{Δ180-226} from colocalizing with and activating cSrc. Thus, we recognized that release of autoinhibition revealed the PH1 domain, which would play a role in facilitating colocalization with cSrc, subsequently enabling AFAP-110 to activate cSrc.

As AFAP-110 contained conserved Arg and Lys residues in the PH1 domain that are predicted to facilitate binding of phospholipids, and there is considerable evidence for cross-talk between PI3K, PKCα and cSrc, we considered that PKCα may be directing activation of PI3K, which in turn may generate a phosphoinositol that could bind to the PH1 domain of AFAP-110 and enable it to colocalize with cSrc upon membranes and subsequently activate cSrc.

To determine if PI3K activity was required for PKCα to direct AFAP-110 to colocalize with and subsequently activate cSrc, the mouse embryo fibroblast system was used. A strength of this model system is that we had matching MEF derived cell lines that had cSrc family kinases knocked out (SYF), cSrc restored (SYF-cSrc) and p85α/β knockouts which resulted in loss of PI3K activity (p85−/−), and a matching cell line with only the p85α subunit expressed (p85α+/+), allowing us to address this question. Pretreatment of cells with two different PI3K inhibitors, as well as deletion of the p85 α/β regulatory subunits of PI3K prevented PMA from directing AFAP-110 to colocalize with and subsequently activating cSrc. Expression of the p85α regulatory subunit of PI3K in the p85−/− cells restored stability of the p110 catalytic subunit of PI3K and restored the ability of PMA to direct AFAP-110 to colocalize with and activate cSrc.

PI3K activity appears to be required for PMA to direct AFAP-110 to colocalize with cSrc and appears to be required for cSrc activation. Because 6 μM bisindolylmaleimide [I] blocks PKCα catalytic activity and blocks subsequent movement of AFAP-110 to cSrc and cSrc activation, we question whether PMA is activating PI3K in a fashion independent of PKCα. Therefore, we recognized that PI3K may function downstream of PKCα.

There is considerable evidence that PI3K can direct activation of PKCα however, there are conflicting data in the literature that sought to determine whether a reverse pathway was active, where PKCα could act upstream and direct activation of PI3K (Balendran, A. et. al., 2000b; Gliki, G. et. al., 2002; Kawakami, Y. et. al., 2004; Kolanus, W. et. al., 1997) (Batendran, A. et. al., 2000a; Balendran, A. et. al., 2000b). To determine whether PI3K could function downstream of PKCα we treated cells with PMA and performed a standard PI3K assay, measuring incorporation of [³²p] into a phospholipid substrate. PMA was able to induce activation of PI3K. To verify this result, we employed the anti-Ptdlns-3,4,5-P₃ antibodies and used immunofluorescence to demonstrate production of Ptdlns-3,4,5-P₃ in cells treated with PMA. These data indicated that both serum induction and PMA were able to direct an increase in Ptdlns-3,4,5-P₃ production. We observed that Ptdlns-3,4,5-P₃ production was consistently higher at 5 minutes post treatment compared to 15 minutes post treatment. Interestingly, we noticed that serum appeared to induce production of Ptdlns-3,4,5-P₃ in both the perinuclear region as well as along the cytoplasmic membrane; whereas PMA only seemed to induce Ptdlns-3,4,5-P₃ production in the perinuclear region of the cell. It is likely that serum contains growth factors that can activate cell surface associated growth factor receptors as well as other cellular signals, while PMA directs activation of a more narrow spectrum of signaling proteins (Barry, O. P. et. al., 2001; Chun, Y. S. et. al., 2003; Fruman, D. A. et. al., 1998; Hai, C. M. et. at., 2002; Kazanietz, M. G., 2000; Kazanietz, M. G. et. al., 2000; Wang, Q. et. al., 1998; Wymann, M. P. et. al., 2003; Wymann, M. P. et. al., 2005). Thus, perhaps serum activates PI3K that exist at the cell periphery and along internal membranes, while PMA may direct activation of a more limited population of PI3K that might localize to internal membranes.

As inactive cSrc is found primarily along perinuclear vesicles (Kaplan, K. B. et. al., 1992; Redmond, T. et. at., 1992), the ability of Ptdlns-3,4,5-P³ to be generated in the perinuclear region of the cell may be consistent with activation of cellular signals which promote cSrc activation. In addition, we noted that PMA could induce an increase in Akt phosphorylation on serine 473 and stabilization of HIF-1α, both surrogate markers for PI3K activation. Thus, our data indicates that PMA can direct PI3K activation in the MEF cell system. This ability to direct PI3K activation is likely dependent upon PKCα, as inhibitors of PKCα catalytic activity block these signals. Also, PKCα is the major PMA inducible PKC family member in these cells and the only PMA-activated PKC family member that can also bind to AFAP-110 (Gatesman, A. et. al., 2004; Qian, Y. et. al., 2002). A mechanism by which PMA can direct PI3K activation is not yet clear. We cannot rule out a role for other PMA inducible PKC family members that are present in this cell system, which could direct PI3K activation, such as PKC5 and PKCε.

We recognize that PKCα, either through constitutive activation (myrPKCα) or subsequent to PMA treatment, can direct activation of PI3K. This indicates that the subsequent generation of Ptdlns-3,4,5-P₃ may play a role in colocalization between AFAP-110 and cSrc by binding to the PH1 domain. This model would be analogous to the mechanism by which Akt becomes activated subsequent to PI3K activation (Burgering, B. M. et. al., 1995; Jiang, B. H et. al., 1999; Klippel, A. et. al., 1997). We sought to determine if the PH1 domain of AFAP-110 had the capacity to bind to phosphatidylinositol or other phospholipids.

We obtained a dot-blot of various phospholipids immobilized on PVDF and incubated it with GST-fusion proteins that expressed the PH domain of DAPP1 as a positive control and GST-PH1 or GST-PH2, then performed a far western blot using anti-GST. Neither GST nor GST-PH2 bound the immobilized phospholipids. This was consistent with our observations that although the PH2 domain has a groove associated with lipid-binding PH domains, it does not have the conserved Arg/Lys residues required to coordinate electrostatic interactions with negatively charged phospho-head groups. GST-DAPP1 is reported to bind to Ptdlns-3,5-P₂ and Ptdlns-3,4,5-P₃ (Dowler, S. et. al., 1999). Incubation of GST-PH1 with this membrane revealed it had the capacity to recognize several phospholipids, including phosphatidylserine, phosphatidic acid and a series of phosphatidylinositols that were phosphorylated on the D-3, D-4 and D-5 positions (either together or separately). GST-PH1 did not bind to phosphatidylinositol, indicating that phosphorylation at the D-3, D-4 and D-5 positions was a requirement for binding.

The structures of the phospholipids that were recognized by the PH1 domain were aligned to consider the structural requirements for binding (FIG. 8). We noted that those phospholipids that were recognized by the PH1 domain of AFAP might also be recognized by the PH1 binding domains of other molecules. Therefore, drugs developed for recognition by AFAP might also affect molecules such as AK1.

Phosphatidic acid (PA) was recognized best by the PH1 domain. This lipid has a small, negatively charged head group and two hydrophobic tails. Lysophosphatidic acid is analogous to PA but has only one hydrophobic tail and was not recognized. Phosphatidylethanolamine is analogous to PA, but has a positively charged NH3+ head group linked with the phospho-group and was not recognized by the PH1 domain. As for the phosphatidylinositols, having one phosphate on the head group promoted binding better than when two phosphate residues were present, while Ptdlns-3,4,5-P₃ bound weakest. In a preferred embodiment, phospholipid docking to the groove in the PH1 domain is optimal when the binding molecule has a small, negatively charged head group and two hydrophobic tails. Using molecular modeling techniques, we were able to confirm that the appropriate Arg/Lys residues were present in the groove could coordinate interactions with negatively charged phosphate head groups.

It is important to note that although a PH domain can recognize an immobilized phospholipid, it may have a different binding spectrum when these phospholipids are incorporated into lipid vesicle membranes. To test this, we incorporated phosphatidytinositols into lipid vesicles, incubated them with GST-PH1 or GST-PH2 and pelleted them in order to determine if the GST-fusion proteins could bind to the vesicles. Our data indicate that GST-PH1 can bind to vesicles that contain Ptdlns-4-P, Ptdlns-5-P, Ptdlns-4,5-P₂ and Ptd-lns-3,4-P₂. Ptdlns-3,4,5-P₃ bound weakly, but this may in part reflect a technical issue, as Ptdlns-3,4,5-P₃ is the most hydrophilic of these phosphatidylinositols and may have partially promoted separation of the vesicles into the aqueous phase during vesicle purification. Interestingly, Ptdlns-3-P did not bind, indicating that perhaps phosphorylation in the 3-position alone may not be sufficient to promote binding to the PH1 domain. Although Ptdlns-3,4,5-P₃ binds to the PH1 domain, it does so weakly.

Immunofluorescence data indicate that Ptdlns-3,4,5-P₃ is produced at significant levels and localizes to perinuclear regions of the cell, where cSrc resides in its inactive state. Thus, perhaps sufficient Ptdlns-3,4,5-P₃ could be produced to bind to the PH1 domain and foster colocalization with cSrc. However, based on the vesicle binding data, a PI3K generated lipid may not be favored to bind over other lipids, as phosphorylation in the 3-position alone did not promote binding. PI3K may be promoting the production of other phospholipids or phosphatidylinositols that bind more favorably. It is interesting that PA bound best on the far western blot. PA has long been suspected to function as a signaling lipid (O'Luanaigh, N. et. al., 2002) and is produced when Phospholipase D processes phosphatidylcholine into PA and choline (Foster, D. A. et. al., 2003). PLD exists in two isoforms, PLD1 and PLD2, with the former being associated with golgi and internal membranes and the latter associated with the cytoplasmic membrane (Liscovitch, et. al, 1999; Xu, L. et. al., 2000). Upon activation, PLD will produce PA which concomitantly is incorporated into golgi membranes (Rizzo, Met. al., 2002; Roth, M. G. et. al., 1997; Roth, M. G. et. al., 1999). In vitro, PA will induce concave curvature in membranes, indicating that it could promote the formation of vesicles. Indeed, when cSrc becomes activated, it moves to the membrane by exocytosis. Interestingly, PLD can be activated by either PKCα or by PI3K. PKCα can bind to PLD and activate it directly (del Peso, L. et. al., 1997; Siddiqi, A. R. et. al., 2000; Yeo, E. J. et. al., 1997), while PI3K can activate PLD and cSrc via an Arf6/Ra1 pathway which promotes exocytosis (Rizzo, M. et. al., 2002; Roth, M G. et. al., 1997; Roth, M. G. et. al., 1999).

Data herein supports a role for PI3K in regulating PKCα directed colocalization of AFAP-110 and cSrc, which in turn is required in order for AFAP-110 to activate cSrc. Our data also indicate that PMA or PKCα can direct activation of PI3K and that the PH1 domain is capable of binding to phospholipids or phosphatidylinositols. Further, mutation of conserved residue Trp169, found to be required for PH domains to promotes bind phosphatidylinositols or phospholipids binding (Shaw, G., 1993), was required for PKCα to direct AFAP-110 to colocalize with and activate cSrc. Therefore, without wishing to be bound by theory, we hypothesize that PI3K directs the formation of a phosphatidylinositol or phospholipid that binds to the PH1 domain and promotes AFAP-110 colocalization with cSrc. Lipids useful in embodiments of the invention include PA as well as those phosphatidytinositols that can bind to the PH1 domain when incorporated into vesicles.

Candidate lipids for use in embodiments of the invention include those having the structure set forth in Formula (I), below:

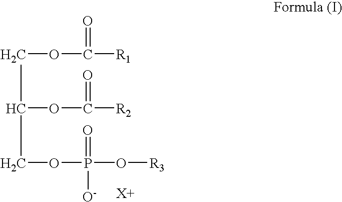

Where R¹ and R₂ are selected independently and represent a linear or branched alkyl group containing 4 to 30 carbon atoms, a linear or branched alkenyl group containing 4 to 30 carbon atoms, or a linear or branched alkynyl group containing 4 to 30 carbon atoms, wherein these groups may comprise a cycloalkane ring or an aromatic ring;

Where R₃ is selected from hydrogen, deuterium, tritium, phosphatidylinositol, phosphatidylinositol-4 phosphate, phosphatidylinositol-5-phosphate, phosphatidylinositol 3-phosphate, a linear or branched alkyl group containing 1 to 4 carbon atoms, a linear or branched alkenyl group containing 2 to 4 carbon atoms, and a linear or branched alkynyl group containing 2 to 4 carbon atoms; and

Where X is selected from hydrogen, an alkali metal atom, and alkali earth metal atom, and a substituted or unsubstituted ammonium group. An alkali metal atom may be, for example, lithium, sodium, potassium, magnesium, or calcium.

In a preferred embodiment, the lipid is selected from the group consisting of phosphatidic acid, phosphatidylinositol-3-phosphate (PI(3)P₁), phosphatidylinositol-4-phosphate (PI(4)P₁), and phosphatidylinositol-5-phosphate (PI(5)P₁). Typically, lipids useful in the invention will have two “tail” groups that are at least C5, and they will have a “head” group that is small and negatively charged.

Other lipids may be useful in embodiments of the invention. These may be lipids having the structure set forth in Formula (II), below:

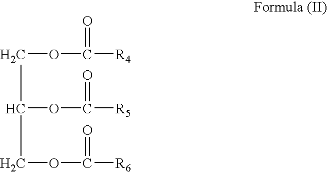

Where R₄ and R₅ are selected independently and represent a linear or branched alkyl group containing 4 to 30 carbon atoms, a linear or branched alkenyl group containing 4 to 30 carbon atoms, or a linear or branched alkynyl group containing 4 to 30 carbon atoms, wherein these groups may comprise a cycloalkane ring or an aromatic ring;

Where R₆ is selected from hydrogen, deuterium, tritium, phosphatidylinositol, phosphatidylinositol-4 phosphate, phosphatidylinositol-5-phosphate, phosphatidylinositol 3-phosphate, a linear or branched alkyl group containing 1 to 4 carbon atoms, a linear or branched alkenyl group containing 2 to 4 carbon atoms, and a linear or branched alkynyl group containing 2 to 4 carbon atoms, chlorine, bromine, fluorine, or iodine.

Examples of the C₄₋₃₀ linear or branched alkyl groups represented by the substituents R₁, R₂, R₄, and R₅, in Formula (I) and Formula (II) include but are not limited to a butyl group, a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, and an eicosyl group.

Examples of the C₄₋₃₀ linear or branched alkenyl group represented by the substituents R₁, R₂, R₄, and R₅ include, for example, but are not limited to a butenyl group, an octenyl group, a decenyl group, a dodecadienyl group, and a hexadecatrienyl group. More specifically, the examples include 8-decenyl group, 8-undecenyl group, 8-dodecenyl group, 8-tridecenyl group, 8-tetradecenyl group, 8-pentadecenyl group, 8-hexadecenyl group, 8-heptadecenyl group, 8-octadecenyl group, 8-icocenyl group, 8-dococenyl group, heptadeca-8,11-dienyl group, heptadeca-8,11,14-trienyl group, nonadeca-4,7,10,13-tetraenyl group, nonadeca-4,7,10,13,16-pentaenyl group, and henicosa-3,6,9,12,15,18-hexaenyl group.

Examples of the C₄₋₃₀ linear or branched alkynyl group represented by substituents R₁, R₂, R₄, and R₅ include, for example, but are not limited to, an 8-decynyl group, 8-undecynyl group, 8-dodecynyl group, 8-tridecynyl group, 8-tetradecynyl group, 8-pentadecynyl group, 8-hexadecynyl group, 8-heptadecynyl group, 8-octadecynyl group, 8-icocynyl group, 8-dococynyl group, and heptadeca-8,11-diynyl group.

Examples of a cycloalkane ring that may be contained in the above described alkyl group, alkenyl group or alkynyl group include, for example, but are not limited to a cyclopropane ring, a cyclobutane ring, a cyclopentane ring, a cyclohexane ring, and a cyclooctane ring. The cycloalkane ring may contain one or more hetero atoms, and examples thereof include an oxylane ring, an oxetane ring, a tetrahydrofuran ring, and an N-methylprolidine ring.

The examples of an aromatic ring which may be contained in the above described alkyl group, alkenyl group or alkynyl group include, for example, a benzene ring, a naphthalene ring, a pyridine ring, a furan ring, and a thiophene ring.

Embodiments of the invention include combinatorial libraries containing candidate lipids, as well as the generation of these combinatorial libraries. Compounds may be synthesized, for example, using methods reported by Rosseto, R., et al. (2006) A new approach to phospholipid synthesis using tetrahydropyranyl glycerol: rapid access to phosphatidic acid and phosphatidylcholine, including mixed-chain glycerophospholipid derivatives. Org Biomol. Chem. 4: 2358-60; and by Shvets, V.I. (1971). Advances in the Synthesis of Glycerol Phosphatide Esters. Russ. Rev. 40(4): 330-45.

Embodiments of the invention include methods for treating individuals who have cancer. Cancer may be, for example, but is not limited to ovarian cancer, breast cancer, esophageal cancer, and intestinal cancer. Embodiments of the invention also include methods for treating individuals who have exhibited a resistance to chemotherapy.

### EXAMPLES

### Example 1

### Materials and Methods

Materials and methods used in preparing the examples of this application were as follows:

Reagents

Dulbecco's modified Eagle's medium (DMEM), Rhodamine (TRITC)-phalloidin, beta actin (β-actin), monoclonal and polyclonal anti-Flag antibodies, and bovine serum albumin (BSA) were purchased from Sigma. Protein A/G PLUS agarose beads and polyclonal cSrc antibody were purchased from Santa Cruz biotechnology. LipofectAMINE were purchased from Invitrogen. Phorbol 12-myristate 13-acetate (PMA), LY294002 (LY), wortmannin (wort) and bisindolylmaleimide [I] (Bis) were obtained from Calbiochem. Monoclonal p85α and p110α antibodies, monoclonal PKCα antibody antibodies were obtained from BD Transduction Laboratories.

The polyclonal AFAP-110 antibodies F1 were generated and characterized as previously described (Qian, Y. et. al., 1999). Monoclonal avian cSrc antibody (EC10) was obtained from Upstate. Phospho-Src family (Tyr416) antibody was purchased from Cell Signaling. Horseradish peroxidase-conjugated anti-rabbit and anti-mouse IgG secondary antibodies, and γ³²P-ATP were obtained from Amersham Biosciences. QuikChange® II XL site-directed mutagenesis kit was obtained from Stratagene, while the AFAP-110^{W169A} primers were purchased from IDT. Phosphatidylinositol (PI) used in the PI3K kinase assay was purchased from Matreya LLC. All Alexa Fluor antibodies used in the paper were purchased from Molecular Probes (Invitrogen). Src-family tyrosine kinase inhibitor, PP1, was purchased from Biomol. Phosphoinositol-3,4,5-trisphosphate (Ptdlns-3,4,5-P₃ or PI-3,4,5-P₃) monoclonal IgM antibody, PIP strips and PIP arrays were obtained from Echelon Biosciences. Phospholipids used in the lipid binding and studies were purchased from Avanti Polar Lipids (Alabaster, Ala.) and Caymen Chemical Company (Ann Arbor Mich.). The peroxidase conjugated goat anti-rabbit IgG secondary antibodies used in the lipid binding assays were purchased from KPL Inc. Chemiluminescence reagent was purchased from Pierce Biochemical. All chemicals used throughout this application, except where otherwise stated, were purchased from J. T. Baker.

Cell Lines and Culture

Mouse embryo fibroblast, SYF/cSrc and SYF (ATCC), cells were used throughout this study. SYF/cSrc are derived from a SYF parental cell line that is devoid of Src family of non-receptor tyrosine kinase membersfyn, and c-yes genes but engineered to re-express cSrc (Klinghoffer, R. A. et. al., 1999). Mouse embryo fibroblasts (p85−/−) derived from Pik3r1 (encodes p85α genes) and Pik3r2 (encodes p85β gene) double knockout or Pik3r2 (p85α+/+) single knockout in 129 C57BL/6 mice were kind gift from Saskia Brachmann (Harvard). The above cell lines were grown at 37° C. with 5% CO₂ in DMEM supplemented with 10% (v/v) fetal calf serum, 1% (v/v) 200 mM L-glutamine and 1% (v/v) penicillin/streptomycin.

Constructs and Transfection

The pEGFP-c3 (green fluorescence protein) expression system from Clontech was used to express GFP-tagged full-length and mutant forms of AFAP-110. AFAP-110 was cloned into this vector as previously described (Qian, Y. et. al., 2000). CMV-1-AFAP-110^{Δ180-226} was previously described (Baisden, J. M. et. al., 2001a). This mutant was cloned into pFlag-CMV-1 from Sigma. Dominant-positive and dominant-negative forms of Flag-tagged PKCα were expressed using the pCMV-1 vectors and were a kind gift from Alex Toker. Avian cSrc was subcloned from Rous Sarcoma Virus (RSV) into pCMV-1 as previously described (Guappone, A. C. et. al., 1996). GFP-tagged AFAP-110^{W169A} was developed by mutating the tryptophan¹⁶⁹ residue to an alanine in full-length GFP-AFAP-110 using the QuikChange® II XL site-directed mutagenesis kit as per manufacturer's protocol. These clones were screened using Ava II restriction enzyme.

Immunofluorescence

SYF, SYF/cSrc, p85α+/+ and p85−/− cells were cultured in standard culture media. Transient transfections of SYF, SYF/cSrc, p85α+/+ cells for immunofluorescence were carried out using LipofectAMINE™ Reagent (Invitrogen) as per manufacturer's protocol. Briefly, Approximately 5.0-8.0×10⁴ cells per well were transfected at 50-70% confluent on coverstips with 2-4 μg of plasmid DNA and incubated for 3-4 hours. Thirty-six hours after transfection, cells were serum starved for 12 hours, treated, and subsequently fixed and permeablized as previously described (Qian, Y. et. al., 1998). Cells were treated with a PKC activator, 100 nM PMA for 5 or 15 minutes or in combination with the following pretreatments; a PKC inhibitor, 6 μM bisindolylmaleimide [I] for six hours; or PI3K inhibitor, 10 μM LY294002 for six hours; 50 nM Wortmannin for 30 minutes; and/or a Src family inhibitor, 5 μM PP1 for six hours. For actin labeling, a 1:500 dilution of TRITC-phalloidin was used as labeled in the figures. Primary antibody concentrations used were diluted in 5% Bovine Serum Albumin (BSA) dissolved in 1× phosphate-buffered saline (PBS): EC10 mAb-1:500; Phospho-Src Family (Y416) pAb-1:250; Anti-PI-3,4,5-P₃ mAb-1:100; Anti-AFAP-110 (F1)pAb-1:1000; Anti-cSrc pAb-1:500; Anti-flag-1:1000. All fluorescent secondary and phalloidin antibodies were diluted 1:200 in 5% BSA, and are labeled according to figure legends. Cells were washed and mounted on slides with Fluoromount-G (Fisher). A Zeiss LSM 510 confocal microscope was used to scan images of about 1 μM in thickness. To prevent cross-contamination between the different fluorochromes, each channel was imaged sequentially using the multitrack recording module before merging the images. For all figures, representative cells are shown (>100 cells examined per image shown).

Immunoblot Assay

All cells were cultured to 70-80% confluence, serum-starved for 16 hours, and treated as described above. The cells were lysed and western analysis performed as previously described (Guappone, A. C. et. al., 1997). Membranes were probed using the following antibodies diluted in 1× Tris-buffered saline plus 0.1% Tween-20 (TBS-T) containing 5% nonfat milk, except were indicated: 1:10000 AFAP-110 pAb (F1), 1:1000 Phospho-Src family Tyrosine 416 (Cell Signal) in 5% BSA, 1:500 cSrc (clone N-16: Santa Cruz Biotechnology), 1:1000 p85α (BD Biosciences), 1:1000 p110α (BD Biosciences), 1:1000 PKCα (BD Biosciences), and 1:5000 β-actin in 1% BSA (Sigma).

PI3K Assay

PI3K activity was determined using the In vitro PI3K kinase assay as previously described (Jiang, B. H. et. al., 1998). Cells were serum-starved 24 hours. The media was changed and cells were then treated with either 10% fetal calf serum or 100 nM PMA for 5 or 15 minutes alone or in conjunction with 6 μM bisindolylmaleimide [i] or 10 μM LY294002 for six hours as a negative control. Upon completion of the incubation, the cells were lysed in cold kinase lysis buffer [150 mM NaCl, 100 mM Tris pH 8.0, 1% Triton X-100, 5 mM EDTA, 10 mM NaF, plus inhibitors (1 mg/ml leuptin, 1 mg/ml peptatin, 0.5 M sodium vanadate, 1 mg/ml aprotinin, and 1M DTT)]. Five hundred micrograms (500 μg) of protein was incubated with p110α antibody overnight at 4° C. Forty microliters (40 μl) of protein A/G PLUS agarose beads (50% slurry) was added and incubated for an additional two hours. The beads were pelleted and washed two times with cold lysis buffer and one time each with fresh cold TNE buffer [20 mM Tris pH 7.5, 100 mM NaCl, and 1 mM EDTA] and once with 20 mM HEPES pH 7.5. The pellets were re-suspended in γ³² P-ATP kinase reaction buffer [20 mM HEPES pH 7.5, 10 mM MgCl₂, 0.2 mg/ml phosphatidylinositol (in 10 mM HEPES pH 7.5) in 10 mM HEPES pH 7.5, 60μ ATP, 0.2 μCi/1μ γ³²P-ATP]. The samples were incubated 15 minutes on ice and the reactions were stopped by adding 1M HCI. Followed by the addition of chloroform/methanol (1:1) the samples were vortexed and the beads pelleted. The lower phase was collected, the samples dried, re-hydrated in chloroform and spotted on a prepared TLC plate. The finished plates were then exposed to radiography film up to 48 hours at −70° C.

Lipid Binding Assay (Far Western)

Pleckstrin homology (PH) fusion proteins GST-PH1, GST-PH2 and GST-DAPP1, were purified from bacteria by affinity chromatography. A Far Western Blot procedure was used for detecting the fusion protein binding to nitrocellulose immobilized phospholipids (Stevenson, J. Met. al., 1998); PIP strips and PIP arrays were purchased from Echelon, Inc. Overlay assays measured the relative amounts of GST-tagged fusion protein bound to spotted phospholipids. A plasmid encoding GST-DAPP1 previously shown to bind Ptdlns-3,4-P₂ and Ptdlns-3,4,5-P₃ (Dowler, S. et. al., 1999) was used as a control, kindly provided by Mark Lemmon (Lemmon, M. A., 2003). The membranes were wetted with methanol and then transferred to blocking solution composed of 3% BSA in TBS containing 1% Tween-20 (TBS-T) for 1 hour at 4° C. Membranes were incubated with 0.5 μg/ml each fusion proteins in TBS-T overnight at 4° C. Following incubation, membranes were rinsed three times with TBS-T, washed for 5 minutes and incubated at 4° C. for 1 hour with anti-GST antibody (1:20000 in TBS-T). Following the standard wash procedure, membranes were incubated at 4° C. for 30 minutes with peroxidase-conjugated secondary antibody (1:30000 in TBS-T). The washed membranes were incubated with chemiluminescence reagent and exposed to X-ray film.

Lipid Sedimentation Assay

A sedimentation assay was used to detect PH domain protein association with membrane phospholipids. Phospholipid stock solutions: 1 mg/ml L-α-Ptdlns-4-P, 1.0 mg/ml 1,2-dipalmitoyl-sn-glycero-3-Ptdlns-3-P, 0.1 mg/ml 1,2-dioleoyl-sn-glycero-3-Ptdlns-3,4-P₂, and 1 mg/ml L-α-Ptdlns-4,5-P₂ in chloroform:methanol:water (60:30:4). However, 10 mg/ml L-α-phosphatidylinositol, 0.1 mg/ml L-α Ptdlns-3-P, 0.2 mg/ml L-α-Ptdlns-3,4,5-P₃, 10 mg/ml L-α-phosphatidylserine, and 10 mg/ml L-α-phosphatidylcholine in chloroform neat. All lipid stocks were purged with dry N₂ and stored −20° C. The lipid vesicles were prepared as follows utilizing a sonication method. Briefly, in a heavy walled tube, PC and another stock phospholipid (e.g. Ptdlns, Ptdlns-3-P, Ptdlns-4-P, Ptdlns-3,4-P₂, Ptdlns-3,5-P₂, Ptdlns-3,4,5-P₃, or PC) were combined in a 19:1 molar ratio; PC and PS were combined in a 7:3 molar ratio. Lipids were dried with a stream of N₂ and traces of solvent were removed by vacuum and resuspended to 5.2 mM PC in buffer B [10 mM HEPES, pH 7.5, 50 mM KCl, 0.5 mM EGTA, and 1.0 mM MgCl₂]. The resulting phospholipid sheets were sonicated to form vesicles. Samples were prepared to contain 1.7 mM vesicle PC and 0.025 mM recombinant GST fusion protein (DAPP1 or PH1) in buffer B. Samples were incubated 60 min and centrifuged with a Beckman Airfuge for 15 min at room temperature. 16% of the supernatant and 100% of the pellet from each sample was used for analysis by Laemmli method of SDS polyacrylamide gel electrophoresis. The gels were stained with SYPRO Orange or Coomassie Blue to visualize the proteins.

### Example 2

### PI3K Activity is Required for PMA-Induced Translocation of AFAP-110 to cSrc and Subsequent cSrc Activation

The ability of AFAP-110 to colocalize with cSrc in response to PMA-directed signals is dependent upon the integrity of the PH1 domain. Deletions in the PH1 domain will prevent PMA or PKCα from inducing AFAP-110 to colocalize with cSrc and will also block PMA or PKCα-directed activation of cSrc. Many PH domains can bind PI3K generated lipids and there is significant evidence for cross talk between PKCα, cSrc and PI3K. Thus, we sought to determine whether PI3K activity was required for PMA-induced translocation of AFAP-110 to cSrc as well as subsequent cSrc activation.

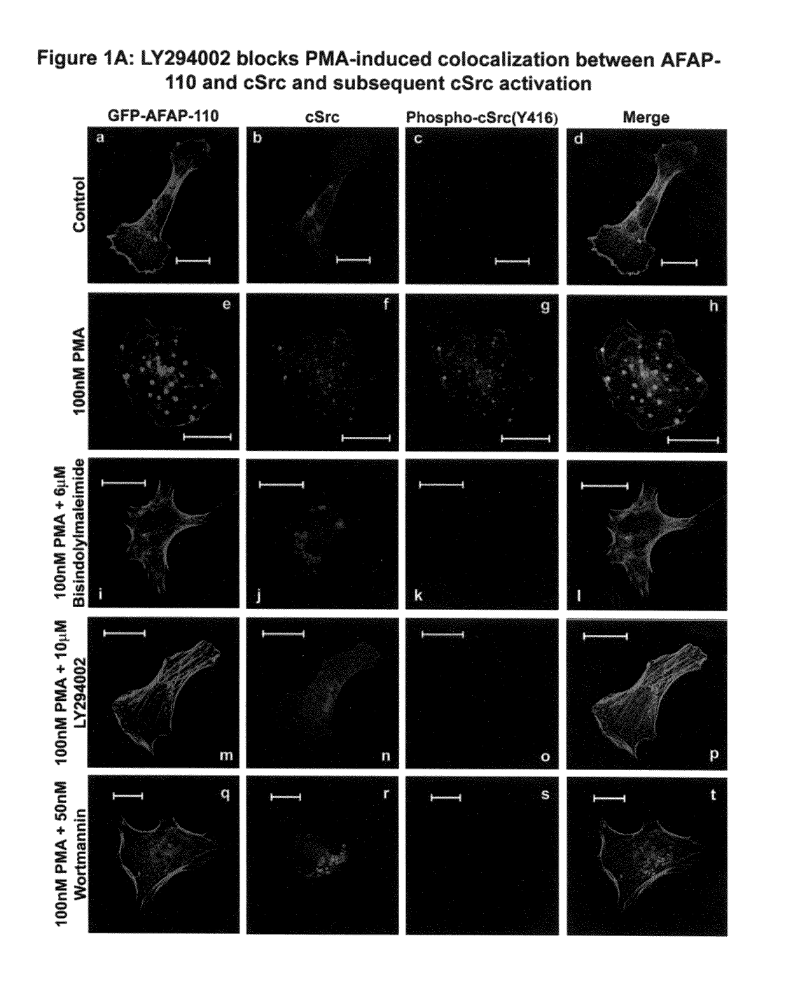

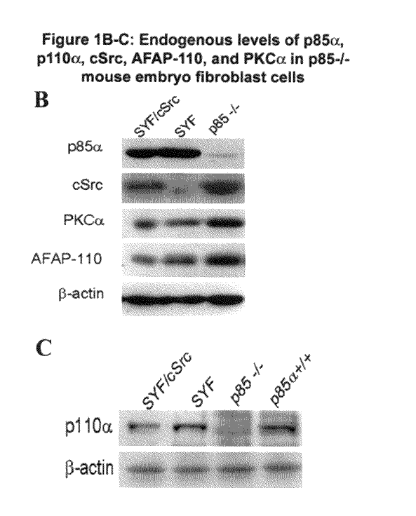

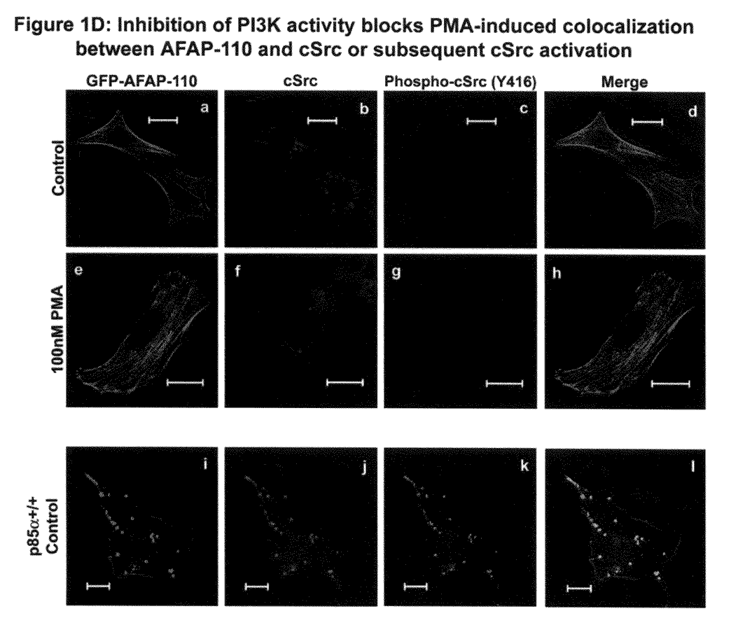

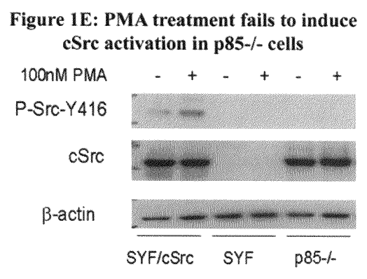

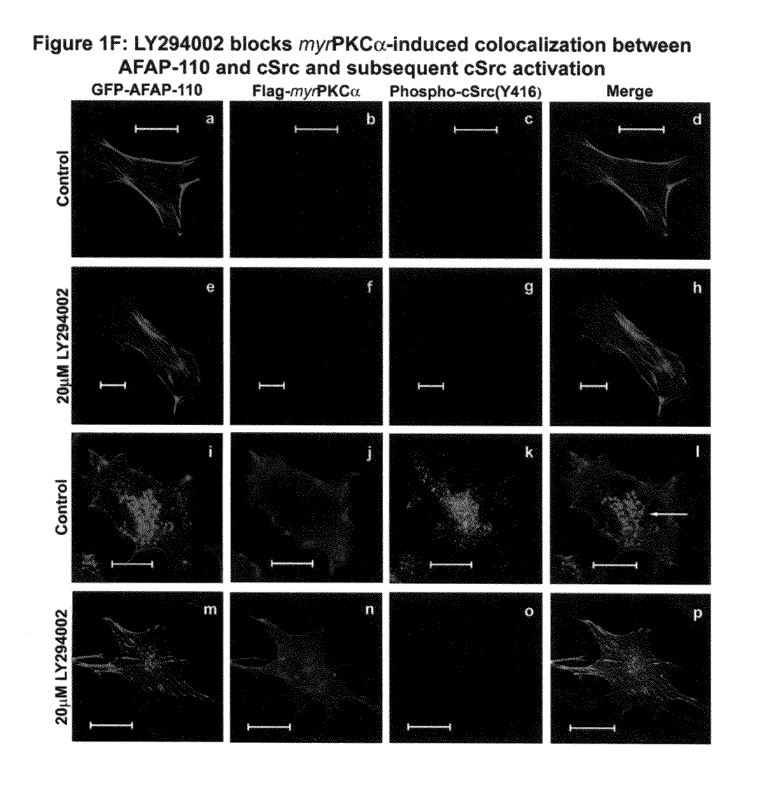

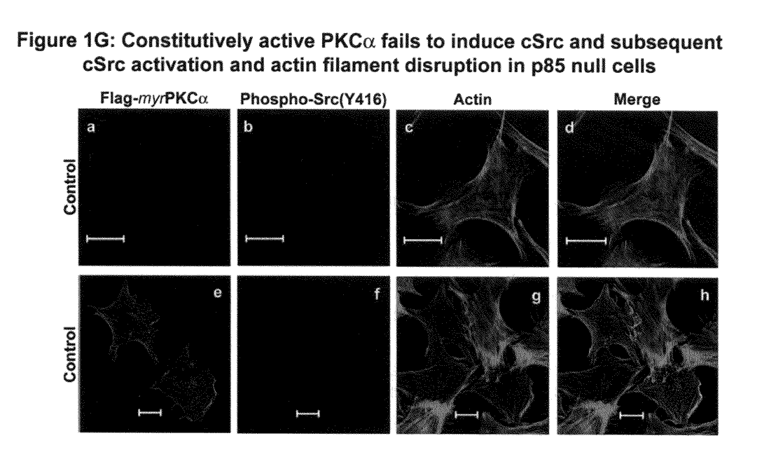

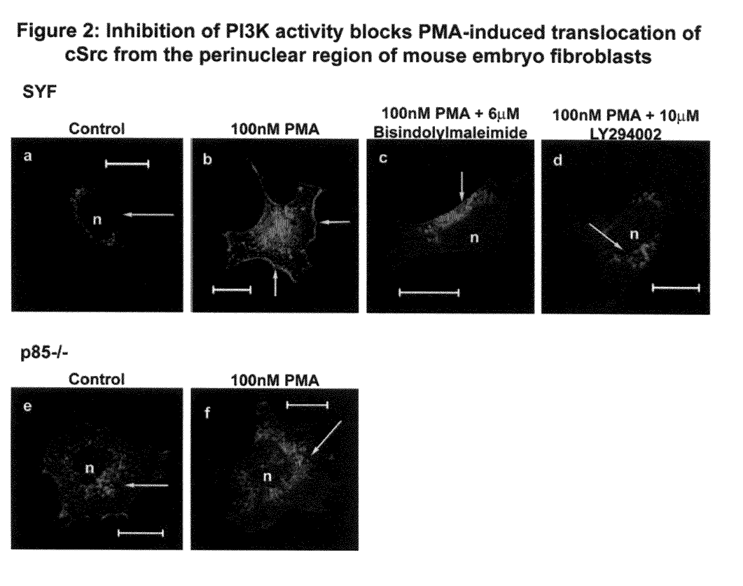

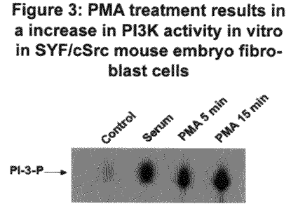

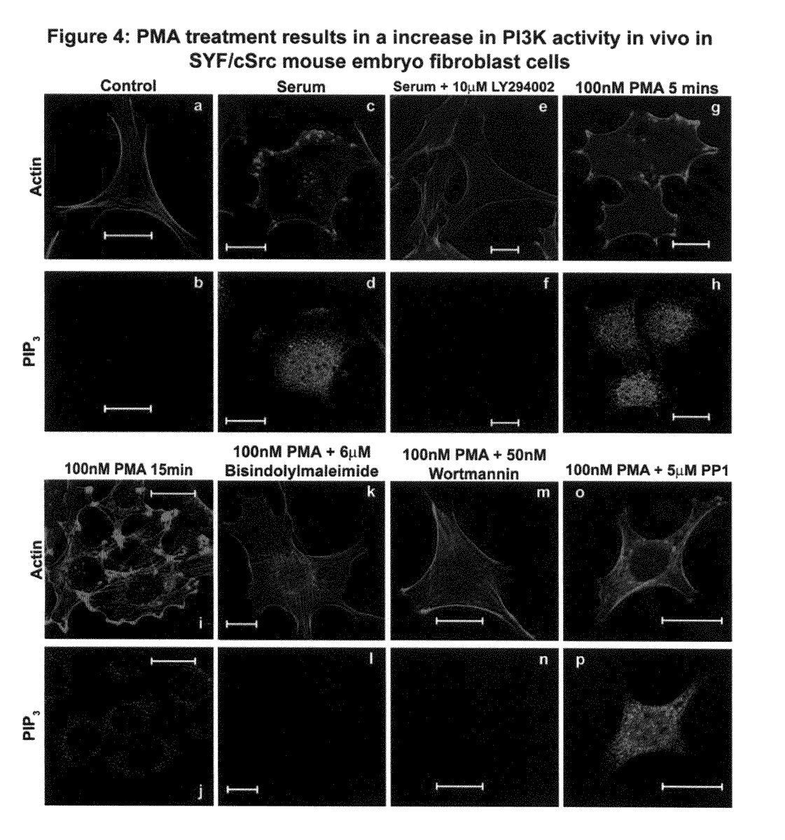

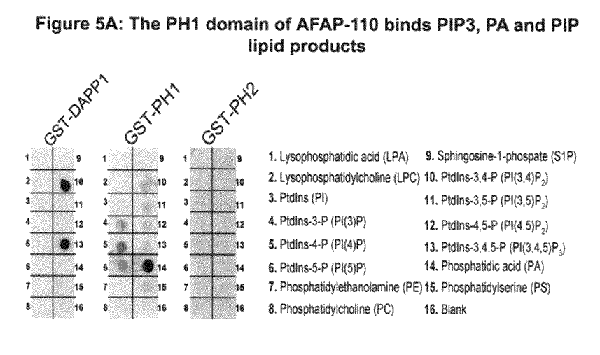

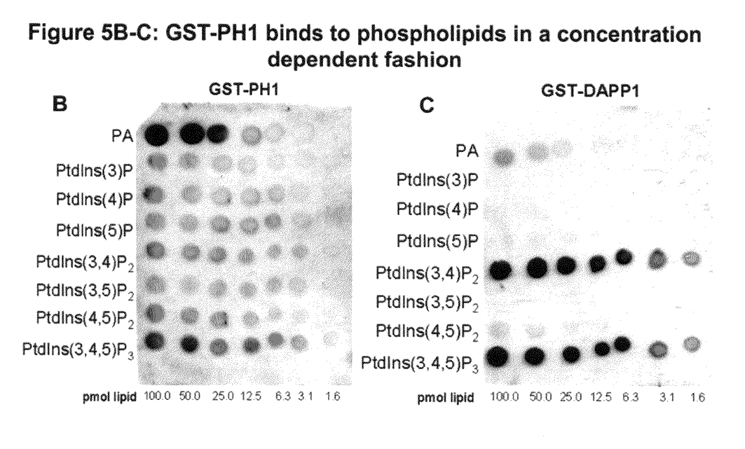

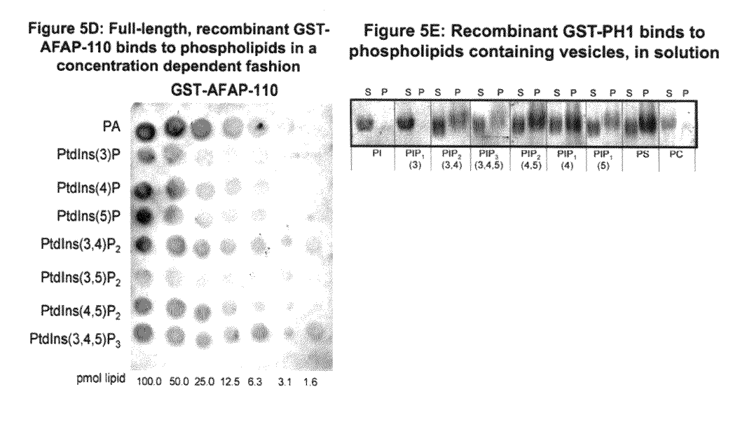

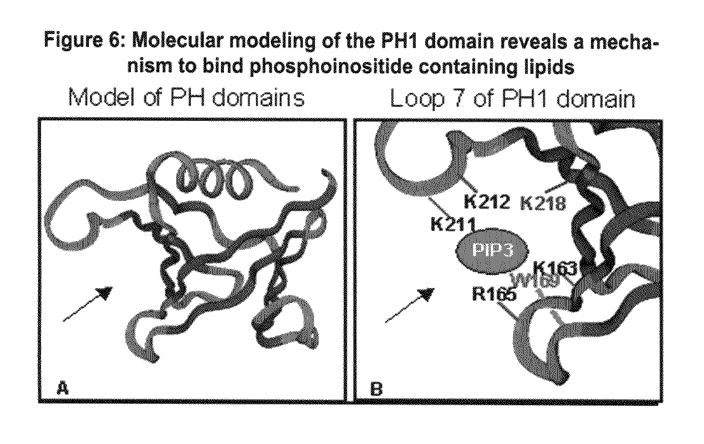

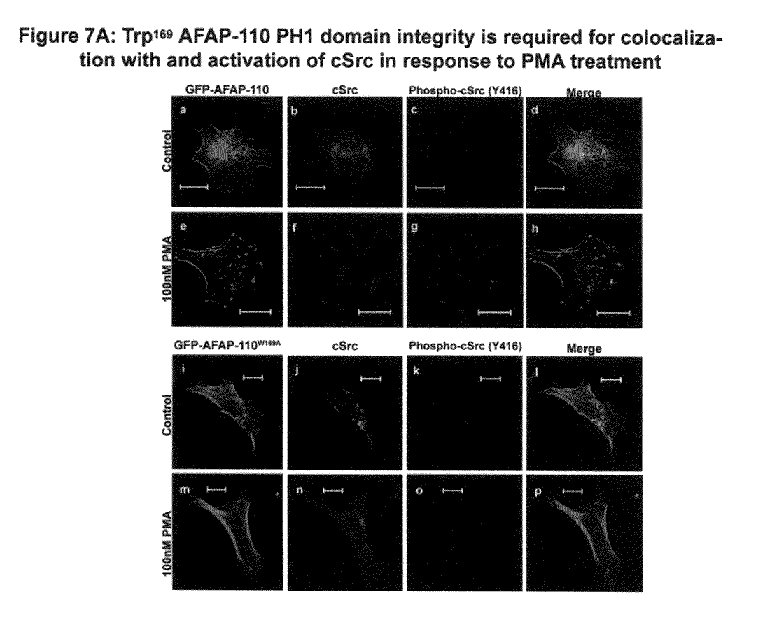

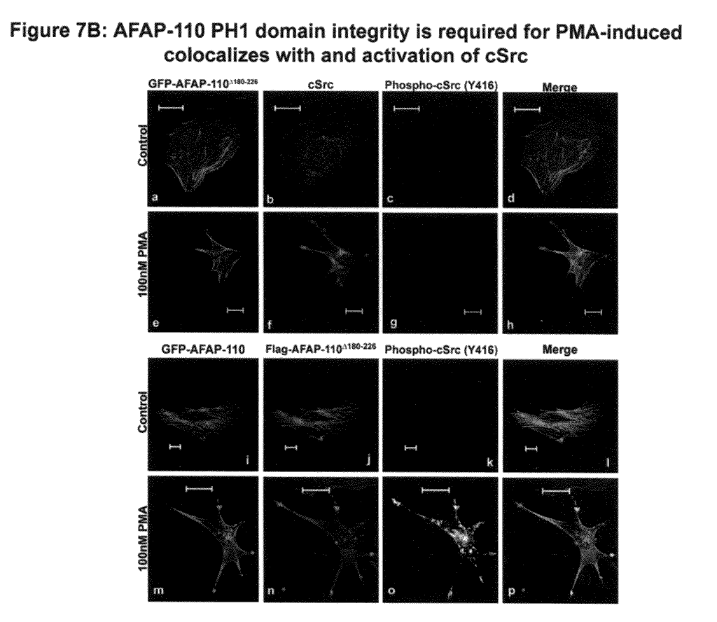

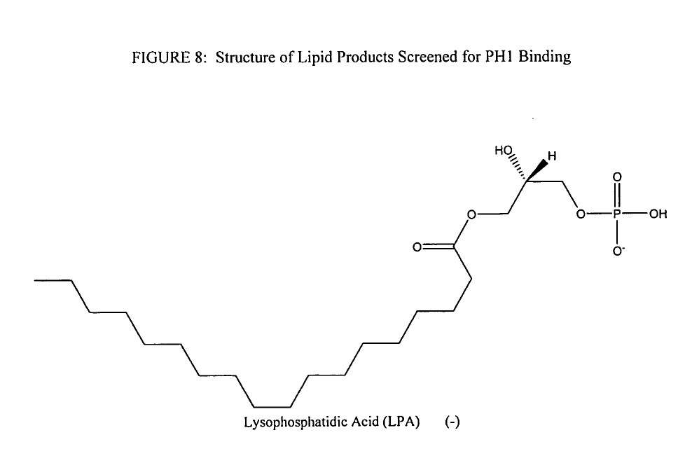

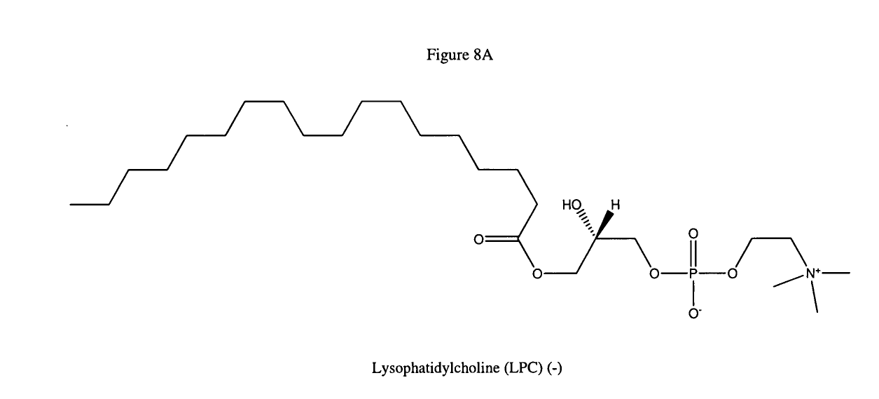

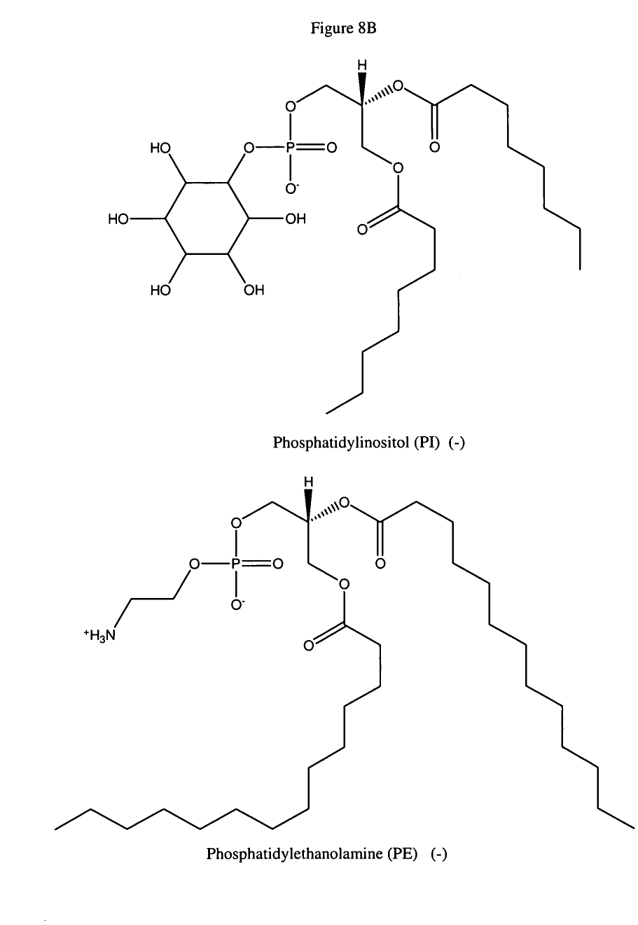

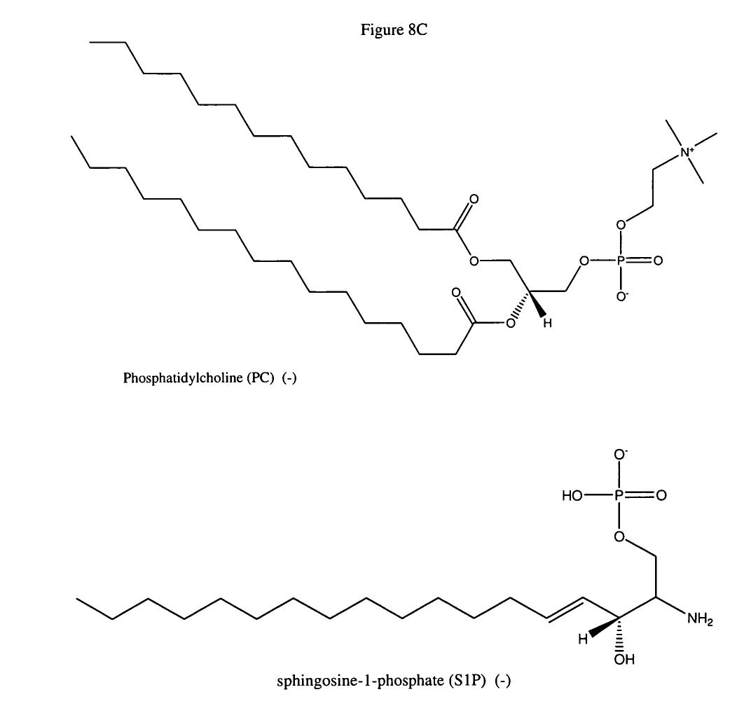

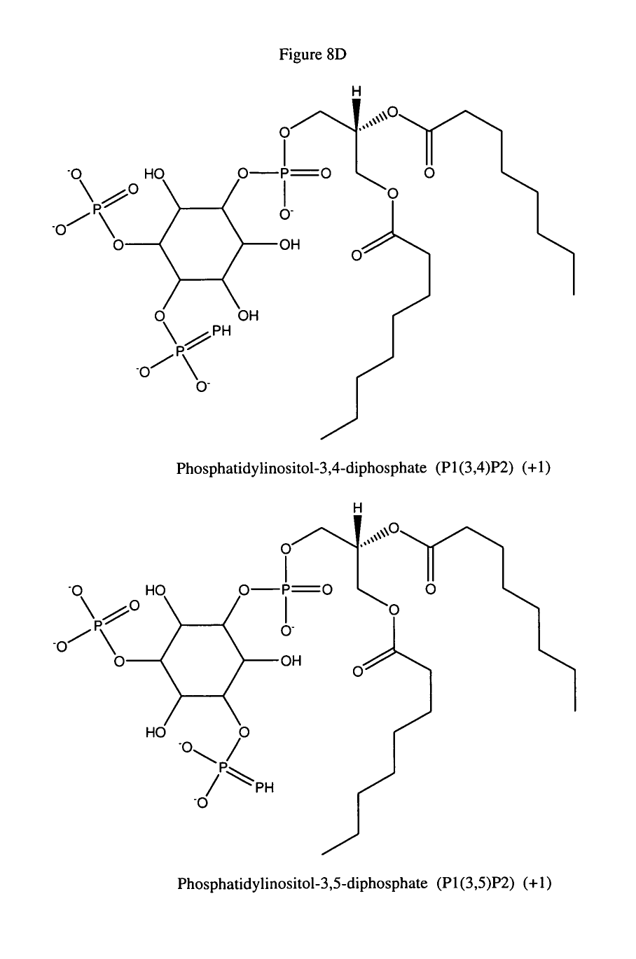

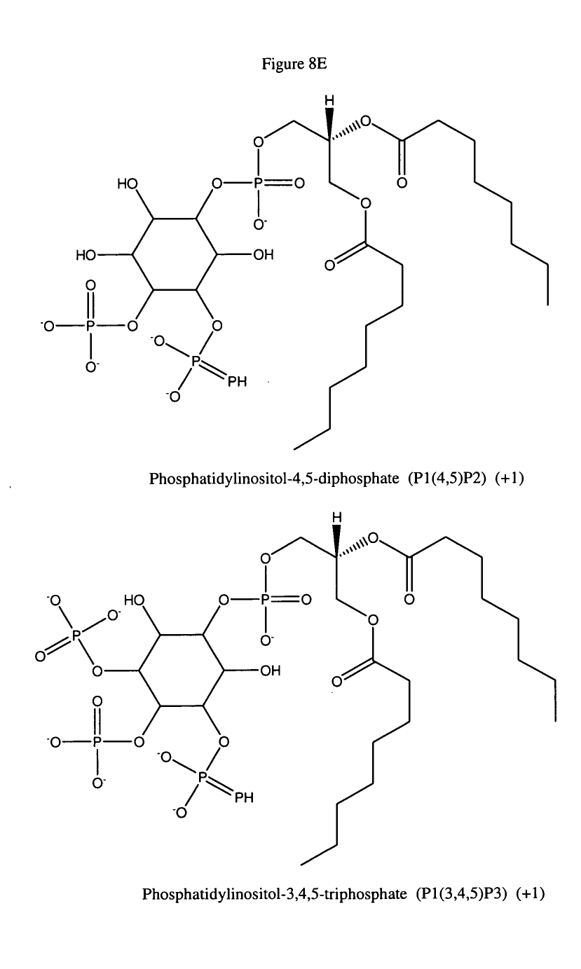

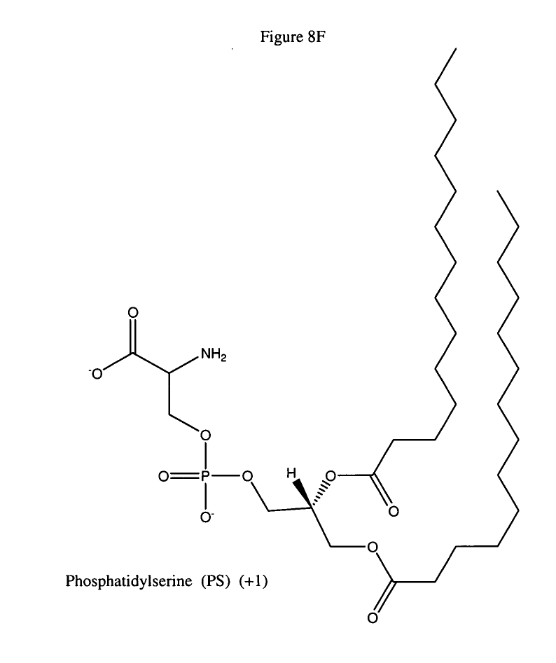

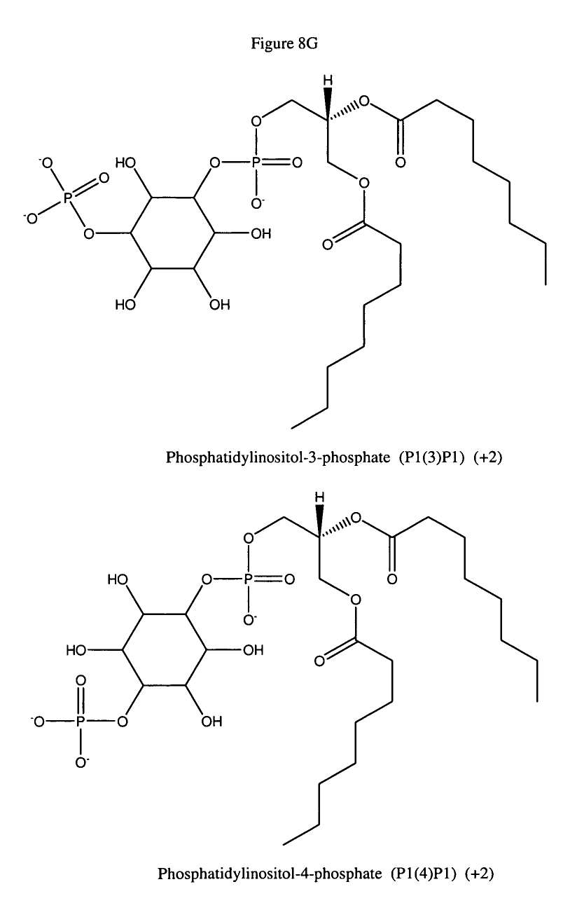

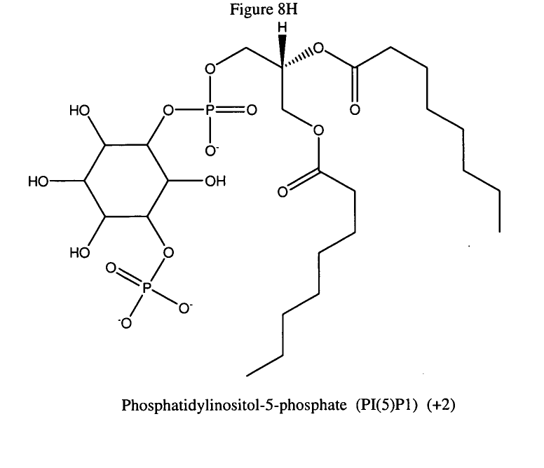

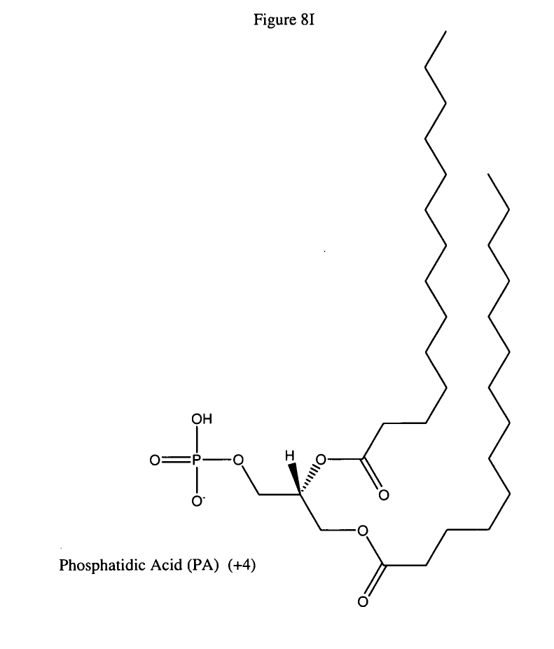

We utilized SYF cell lines as a model system, mouse embryo fibroblasts engineered to contain null mutations in the c-src,fyn, and c-yes genes (Klinghoffer, R. A. et. al., 1999). The cells were transiently co-transfected with constructs that encode cSrc and GFP-tagged AFAP-110, as previously described (Gatesman, A. et. al., 2004). It has been well demonstrated that over-expression of these proteins do not alter their cellular location (Qian, Y. et. al., 2000; Reynolds, A. B. et. al., 1989). Co-expression of GFP-AFAP-110 and cSrc in unstimulated SYF cells confirm that wild-type AFAP-110 neither colocalizes with nor activates cSrc (FIG. 1A, panels a-d). PMA treatment caused GFP-AFAP-110 to colocalize with cSrc and there was evidence for activation of cSrc, based on increased immunoreactivity with the anti-phospho-cSrc (Y416) antibody, which recognizes cSrc in its activated state (FIG. 1A, panels e-h). Further, activation of cSrc corresponded with morphological changes associated with the formation of dot-like structures on the ventral membrane, which are consistent with podosomes or invadopodia. Pre-treatment of cells with the PKC inhibitor, bisindolytmaleimide [I] (FIG. 1A, panels i-I) or the PI3K inhibitor, LY294002 (FIG. 1A, panels m-p) blocked PMA-induced colocalization of AFAP-110 with cSrc, cSrc activation and associated morphological changes.

To confirm the specificity of LY294002, cells pre-treated with the PI3K inhibitor, Wortmannin, which also blocked colocalization and cSrc activation (FIG. 1A, panels q-t), and verify these findings (Gatesman, A. et. al., 2004). These data indicate a potential role for PI3K activation in modulating colocalization between AFAP-110 and cSrc. To test the requirement of PI3K, we used a p85 α/β knockout (p85−/−) mouse embryo fibroblast cells (Brachmann, S. M. et. al., 2005; Fruman, D. A. et. al., 2000; Ueki, K. et. al., 2002). We sought to determine the steady state levels of expression of endogenous PKCα or the p110 catalytic subunit of PI3K in our MEF model system. Western blot analysis indicates that p85−/− cells contain significant endogenous levels of PKCα, AFAP-110 and cS rc relative to SYF or SYF/cSrc cells (FIG. 1B). Yu et. al. reported that the regulatory subunit is required to stabilize the catalytic subunit of PI3K (Yu, J. et. al., 1998). Our results support that observation and indicate that p85−/− cells lack detectable levels of p110α, while the p110α subunit is stably expressed when the p85α regulatory subunit of PI3K is present (p85α+/+) in these cells (Figure lC). The p85−/− cells were transiently co-transfected with a GFP-tagged wild-type AFAP-110 and cSrc and evaluated for the ability of AFAP-110 to colocalize with and activate cSrc. PMA failed to induce significant colocalization of AFAP-110 with cSrc (FIG. 1D, panel h) and there was no evidence for cSrc activation in PMA treated p85−/− cells (FIG. 1D, panel g). The role of the p85 regulatory subunit in PMA-induced cSrc activation was confirmed by utilizing the p85α+/+, which express the p85α regulatory subunit. These cells were also transiently transfected with GFP-tagged AFAP-110 and cSrc in the presence or absence of PMA treatment.

Expression of p85α enables PMA to induce colocalization of AFAP-110 with cSrc (FIG. 1D panel I) and cSrc activation (FIG. 1D panel k). Western blot analysis was performed to verify the ability of PMA treatment to activate cSrc. As predicted, PMA induced tyrosine⁴¹⁶ phosphorylation of cSrc in SYF/cSrc and not cells lacking cSrc (SYF) (FIG. 1E). Furthermore, PMA failed to induce cSrc activation in the mouse embryo fibroblast that tack the p85 regulatory subunit. These results further support a role for PI3K in PMA-induced cSrc activation. In order to verify that the effects observed with PMA treatment were associated with PKCα signaling, SYF/cSrc cells were transiently transfected with a constitutively active form of PKCα (myrPKCα). SYF/cSrc cells were co-transfected with GFP-AFAP-110 with or without myrPKCα and examined for cSrc activation.

Expression of GFP-tagged AFAP-110 in the absence of myrPKCα did not direct increased cSrc activation (FIG. 1F panel c). LY294002 had little effect on localization (FIG. 1F panels e-h). Co-expression of AFAP-110 with myrPKCα resulted in an increase in cSrc activation (FIG. 1F panel k) concomitant with significant changes in actin filament integrity (FIG. 1F panel i). Inhibition of PI3K activity by LY294002 blocked the ability of myrPKCα to induce cSrc activation or changes in stress filament integrity (FIG. 1F panels m and o, respectively). To further evaluate the role of PI3K in PKCα induced PMA-directed cSrc activation and actin filament disruption, p85−/− cells were transfected with myrPKCα. Expression of myrPKCα failed to result in an increase of activation of cSrc or promote disruption of actin filaments (FIG. 1G, panels b and c, respectively) relative to untransfected controls (FIG. 1G, panels f and g, respectively). These data indicate that PMA or myrPKCα are unable to direct AFAP-110 to colocalize with or activate cSrc in the absence of the PI3K activity.

In the quiescent state, the vast majority of cSrc is found associated in the perinuclear region of the cell and this location was shown to correlate with an association with perinuclear vesicles both in endogenous and over-expression systems (Kaplan, K. B. et. al., 1992; Redmond, T. et. al., 1992; Reynolds, A. B. et. al., 1989; Sandilands, E. et. al., 2004). Upon activation, cSrc moves to the peripheral cell membrane and stimulates phosphorylation of proteins and downstream signaling cascades that regulate mitogenesis, motility and invasive potential (Sandilands, E. et. al., 2004). Our analysis of cSrc in quiescent cells confirmed that the vast majority of cSrc is associated with the perinuclear region of the cell; while far less is associated with the cell periphery (FIG. 2, panel a). Activation of PKCα by PMA induced cSrc to translocate from the perinuclear region to the cell periphery (FIG. 2, panel b). This movement was blocked by pre-treatment of cells with either bisindolytmaleimide [I] (FIG. 2, panel c) or LY294002 (FIG. 2, panel d). Interestingly, in the absence of PI3K activity cSrc remained in the perinuclear region in p85−/− cells irrespective of PMA treatment (FIG. 2, panels e and f). These data confirm that PI3K activation is also required for PMA induced cSrc translocation to the cell periphery.

### Example 3

### PMA Induces PI3K Activation

Our data indicated that PI3K activity is downstream of PMA treatment and upstream of cSrc activation. Therefore, we predicted that PMA should be able to induce activation of PI3K. Although there are several reports in the literature that indicate PI3K activation can lead to subsequent PKC activation (as a result of PDKI phosphorylation) (Balendran, A. et. al., 2000b); it was not clear whether PMA and PKCα activation can function as upstream activators of PI3K. To test this, we performed a PI3K lipid kinase activity assay (Jiang, B. H. et. al., 1998). SYF/cSrc cells (FIG. 1B) were serum-starved and then stimulated with either serum or PMA for 5 or 15 minutes. The lipid kinase assay demonstrates that there is a significant increase in PI3K activity after 5 or 15 minutes of treatment with PMA (FIG. 3).

To corroborate our results observed in vitro, we stimulated SYF/cSrc cells with PMA and measured the production of Ptdlns-3,4,5-P₃, the predominant lipid product generated by PI3K upon activation. Ptdlns-3,4,5-P₃ production was measured using the anti-Ptdlns-3,4,5-P3 antibody (Echelon, inc.) for analysis by immunofluorescence and contrasted with stress filament integrity, measured using TRITC-phalloidin as previously described (Chen, R. et. al., 2002; Hama, H. et. al., 2004). SYF/cSrc cells were left untreated or treated with serum as controls for PI3K activation. Serum was able to direct upregulation of Ptdlns-3,4,5-P₃ (Katso, R. et. al., 2001) and the PI3K inhibitor LY294002 was able to block serum-induced Ptdlns-3,4,5-P₃ production (FIG. 4, panels c-f). In addition, SYF/cSrc cells were stimulated with PMA for 5 or 15 minutes. The data indicate that PMA was able to induce Ptdlns-3,4,5-P₃ production within 5 minutes of treatment concomitant with significant changes in cell shape and actin filament organization (FIG. 4, panels g and h). Interestingly, Ptdlns-3,4,5-P₃ levels were consistently reduced after 15 minutes of treatment with PMA, although cell shape changes and actin filament reorganization were still apparent (FIG. 4, panels i and j). Pretreatment with either bisindolylmaleimide [I] or wortmannin blocked PMA-induced Ptdlns-3,4,5-P₃ production (FIG. 4, panels k-n) and similar results were observed with LY294002 (data not shown). Based on these results, we predicted that PKC(X and PI3K signaling were upstream of cSrc in response to PMA treatment. To verify this result, cells were treated with the cSrc inhibitor, PP1, which did not block PMA-induced Ptdlns-3,4,5-P₃ production (FIG. 4, panels o and p), although PMA-directed changes in stress filaments and cell shape were impeded. Collectively, these data indicate that PMA treatment induces PKCa-dependent activation of PI3K.

### EXAMPLE 4

The AFAP-110 PH1 domain can bind to phosphoinositides.

Our data indicate that PMA can direct activation of PI3K, and PI3K activity is required for AFAP-110 to move to and activate cSrc. The ability of AFAP-110 to move to cSrc in response to PMA is dependent upon the integrity of the amino terminal pleckstrin homology (PH1) domain (Baisden, J. M. et. al., 2001a; Gatesman, A. et. al., 2004). AFAP-110 contains two PH domains, one amino terminal (PH1) and the other carboxy terminal (PH2) to the predicted PKCα phosphorylation sites. PH domains are modular domains known to bind both proteins and lipids (Lemmon, M. A., 2003). Therefore, we sought to determine whether the PH 1 domain of AFAP-110 could bind to different phospholipids and phosphoinositides, in vitro. Phospholipids and phosphoinositides that were immobilized on membranes were probed with GST-PH1, GST-PH2 or GST-DAPP1, the latter being a PH domain known to bind to PI3K generated lipid products Ptdlns-3,5- P₂ and Ptdlns-3,4,5- P₃ (Dowler, S. et. al., 1999). After incubation of the membranes with GST-fusion proteins that encode these three PH domains, the membrane was probed with anti-GST by far western blot to determine the level of binding (FIG. 5A). The data demonstrate that DAPP1 binds well to Ptdlns-3,5-P₂ and Ptdlns-3,4,5-P₃. The amino terminal GST-PH1 demonstrated strong binding to Ptdlns-3-P, Ptdlns-4-P, Ptdlns-5-P, Ptdlns-3,4-P₂, Ptdlns-3,5-P₂, and Ptdlns-4,5-P₂, with Ptdlns-3,4,5-P₃ binding the weakest. Interestingly, GST-PH1 bound phosphatidic acid (PA) better than any other phospholipid analyzed.

The more carboxy terminal PH2 domain failed to recognize these lipid products. To validate these results, the far western was repeated using several concentration of each lipid (FIG. 5B) and compared to DAPP1 (FIG. 5C). These results support the previous figure and show that the PH1 domain binds the same lipid products in a concentration dependent fashion. In addition, full-length recombinant AFAP-110 bound these same lipids in a concentration dependent fashion (FIG. 5D). While the Far western blot is useful for revealing the capacity of a phospholipids and phosphoinositides to bind, it does not indicate whether the PH domains can bind to phospholipids or phosphoinositides in solution. To test this, phospholipid vesicles were generated that incorporated the same phosphoinositides analyzed in FIG. 5A. The vesicles were incubated with the GST fusion proteins and pelleted using a sedimentation assay.

If GST-fusion proteins bind to vesicles, they will co-sediment with the pellet fraction (P), wile lack of binding will partition them with the supernatant fraction (S). S and P fractions were resolved by SDS-PAGE and GST-fusion proteins detected by Coomassie stain (FIG. 5E). The data demonstrate that GST-PH1 has the capacity to bind to lipid vesicles that contain phosphatidylserine (PS), Ptdlns-4-P, Ptdlns -5-P, Ptdlns-3,4-P₂, Ptdlns-4,5-P₂. Binding to Ptdlns-3,4,5-P₃ was low; however, that may reflect a technical problem with the experiment, as Ptdlns-3,4,5-P₃ is the most water-soluble of the lipids examined and may have partitioned with the aqueous phase during purification of lipid vesicles. These data indicate that the PH1 domain of AFAP-110 is capable of binding to phosphoinositides in solution and may indicate a potential mechanism by which PI3K regulates AFAP-110 dependent activation of cSrc in response to PMA.

### Example 5

### Trp 169 in the PH1 Domain is Required for PMA to Direct AFAP-110 to Colocalize with cSrc, and Subsequently Activate cSrc

Using molecular modeling techniques, we had determined that the PH1 domain could contain a groove consistent with a lipid docking site (Baisden, J. Met. al., 2001b). Although many PH domains contain an analogous groove, not all PH domains bind phospholipids. Phospholipid binding is dependent upon the presence of positively charged amino acids that can form electrostatic interactions with the negatively charged head groups of phospholipids (Thomas, C. C. et. al., 2001; Thomas, C. C. et. al., 2002). To determine if the integrity of the lipid binding pocket is specifically required for AFAP-110 to colocalize with cSrc in response to PMA, we analyzed the groove to determine if positively charged Arginine (Arg) or Lysine (Lys) residues were conserved, which would coordinate electrostatic interactions with negatively charged phospho-head groups (Thomas, C. C. et. al., 2001; Thomas, C. C. et. al., 2002).

Using molecular modeling techniques, we discerned that the PH1 domain contains five Arg/Lys residues in the loop and beta sheets of this groove that are conserved among PH domains that bind to phospholipids or phosphoinositides (FIG. 6). Further, we used the ScanSite program (http://scansite.mit.edu) to determine whether other key residues in the PH1 domain may be required for lipid binding (Obenauer, J. C. et. al., 2003) (Alberti, S., 1998; Isakoff, S. J. et. al., 1998). ScanSite analysis indicated that Trp¹⁶⁹ was a strongly conserved residue within the PH1 that was required for lipid binding (L. Cantley, personal communication) (Gibson, T. J. et. al., 1994; Macias, M J. et. al., 1994; Musacchio, A. et. al., 1993; Yoon, H. S. et. al., 1994).

This tryptophan is believed to act as a stabilizer for PH domains, and may form hydrogen bonds with the head groups of phospholipids (Hyvonen, M. et. al., 1995; Petersen, F. N. et. al., 2005; Zheng, Y. et. al., 1996; Zheng, Y., 2001). Based on the molecular model, Trp¹⁶⁹ is positioned towards the binding pocket and is hypothesized to associate with the phospholipid head group similar to the Trp²³of β-spectrin (Baisden, J. M. et. al., 2001b; Ferguson, K. M. et. al., 2000; Gibson, T. J. et. al., 1994; Hyvonen, M. et. al., 1995; Lemmon, M A. et. al., 2000). Interestingly, the PH2 domain does not have these conserved positively charged residues within this groove possibly explaining the lack of lipid binding (Clump and Flynn, manuscript in preparation).

We engineered in a point mutation, changing Trp69→Ala¹⁶⁹, expressed this construct as a GFP fusion protein in cells, and then treated with PMA to determine if it could colocalize with or activate cSrc. As previously demonstrated, PMA treatment of cells expressing wild-type AFAP-110 resulted in an increase in cSrc activation and a marked disruption of actin filament integrity (FIG. 7A, panels e-h) as compared to untreated controls (FIG. 7A, panels a-d). PMA was unable to induce colocalization between AFAP-110^{169A} and cSrc or activation of cSrc (FIG. 7A, panels m-p). These data indicate that Trp¹⁶⁹, which is conserved among PH domains that bind phospholipids or phosphoinositides was required for AFAP-110 to colocalize with and subsequently activate cSrc in response to PMA.

Deletion of amino acids 180-226 from the PH1 domain render it non-functional and AFAP-110^{Δ180-226} is unable to colocalize with or activate cSrc in response to PMA treatment or PKCα activation (Baisden, J. M. et. al., 2001b); (Gatesman, A. et. al., 2004). This deletion likely has a significant affect on the overall structure of the PH domain. Indeed, AFAP^{Δ180-226} did block PMA induced cSrc activation and was unable to colocalize with cSrc (FIG. 7B, panels a-h) behaving in a similar manner to the Trp^{169A} mutant. To determine if this PH1 domain deletion mutant was acting as a dominant negative we asked if AFAP-110 and AFAP^{Δ180-226} could colocalize and whether over-expression of AFAP-110 could compensate for the ability of AFAP-110^{Δ180-226} to block PMA induced colocalization and cSrc activation. SYF/cSrc cells were co-transfected with a GFP-tagged wild-type AFAP-110 and the flag-tagged PH1 domain was deleted (AFAP-110^{Δ180-226}).

The data reveal that AFAP-110 and AFAP-110^{Δ180-226} do largely colocalize (FIG. 7B, panels i-l). Interestingly we consistently noticed regions along the membrane at the trailing edge where AFAP-110^{Δ180-226} could be found, but AFAP-110 was not found (FIG. 7B, panel 1). Treatment of these cells with PMA was able to direct cSrc activation, indicating that the over-expressed wild type AFAP-110 could compensate for the activity of AFAP-110^{Δ180-226}, the latter of which was likely acting as a competitive inhibitor of cSrc activation in response to PMA.

All claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, and the claims referred to above including but not limited to any original claims.

As used herein and in the appended claims, the singular forms “a,” “an,” and “the” include plural reference unless the context clearly dictates otherwise.

The terms and expressions employed herein have been used as terms of description and not of limitation, and there is no intention in the use of such terms and expressions, or any portions thereof, to exclude any equivalents now know or later developed, whether or not such equivalents are set forth or shown or described herein or whether or not such equivalents are viewed as predictable, but it is recognized that various modifications are within the scope of the invention claimed, whether or not those claims issued with or without alteration or amendment for any reason. Thus, it shall be understood that, although the present invention has been specifically disclosed by preferred embodiments and optional features, modifications and variations of the inventions embodied therein or herein disclosed can be resorted to by those skilled in the art, and such modifications and variations are considered to be within the scope of the inventions disclosed and claimed herein.

Specific methods and compositions described herein are representative of preferred embodiments and are exemplary and not intended as limitations on the scope of the invention.

Other objects, aspects, and embodiments will occur to those skilled in the art upon consideration of this specification, and are encompassed within the spirit of the invention as defined by the scope of the claims. Where examples are given, the description shall be construed to include but not to be limited to only those examples. It will be readily apparent to one skilled in the art that varying substitutions and modifications may be made to the invention disclosed herein without departing from the scope and spirit of the invention, and from the description of the inventions, including those illustratively set forth herein, it is manifest that various modifications and equivalents can be used to implement the concepts of the present invention without departing from its scope. A person of ordinary skill in the art will recognize that changes can be made in form and detail without departing from the spirit and the scope of the invention. The described embodiments are to be considered in all respects as illustrative and not restrictive. Thus, for example, additional embodiments are within the scope of the invention and within the following claims.

We claim:

1. A method for inhibiting cSrc activation, comprising treating cells with a composition comprising a compound selected from the group consisting of compounds of Formula (I): 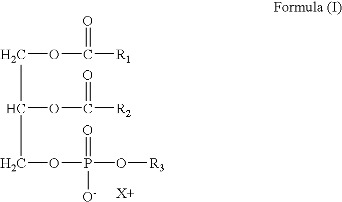
wherein R¹ and R² are selected independently and represent a linear or branched alkyl group containing 4 to 30 carbon atoms, a linear or branched alkenyl group containing 4 to 30 carbon atoms, or a linear or branched alkynyl group containing 4 to 30 carbon atoms, wherein these groups may comprise a cycloalkane ring or an aromatic ring;
wherein R³ is selected from hydrogen, deuterium, tritium, phosphatidylinositol, phosphatidylinositol-4 phosphate, phosphatidylinositol-5-phosphate, phosphatidylinositol 3-phosphate, a linear or branched alkyl group containing 1 to 4 carbon atoms, a linear or branched alkenyl group containing 2 to 4 carbon atoms, and a linear or branched alkynyl group containing 2 to 4 carbon atoms; and
wherein X is selected from hydrogen, an alkali metal atom, and alkali earth metal atom, and a substituted or unsubstituted ammonium group;
its pharmaceutically acceptable esters, and its pharmaceutically acceptable salts;
wherein said compound inhibits cSrc activation.

2. The method of claim 1, wherein said compound is a member of the group consisting of phosphatidic acid, phosphatidylinositol-3-phosphate (PI(3)P₁), phosphatidylinositol-4-phosphate (PI(4)P₁), and phosphatidylinositol-5-phosphate (PI(5)P₁).

3. A pharmaceutical composition comprising a cSrc inhibitor of the general formula of Formula (I), below: 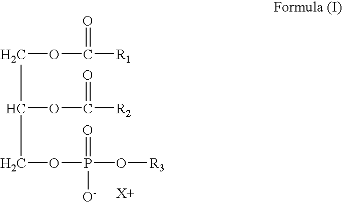
wherein R₁ and R₂ are selected independently and represent a linear or branched alkyl group containing 4 to 30 carbon atoms, a linear or branched alkenyl group containing 4 to 30 carbon atoms, or a linear or branched alkynyl group containing 4 to 30 carbon atoms, wherein these groups may comprise a cycloalkane ring or an aromatic ring;
wherein R₃ is selected from hydrogen, deuterium, tritium, phosphatidylinositol, phosphatidylinositol-4 phosphate, phosphatidylinositol-5-phosphate, phosphatidylinositol 3-phosphate, a linear or branched alkyl group containing 1 to 4 carbon atoms, a linear or branched alkenyl group containing 2 to 4 carbon atoms, and a linear or branched alkynyl group containing 2 to 4 carbon atoms; and
wherein X is selected from hydrogen, an alkali metal atom, and alkali earth metal atom, and a substituted or unsubstituted ammonium group;
its pharmaceutically acceptable esters, and its pharmaceutically acceptable salts.

4. The pharmaceutical composition of claim 3, wherein said pharmaceutical composition is selected from the group consisting of phosphatidic acid, phosphatidylinositol-3-phosphate (PI(3)P₁), phosphatidylinositol-4-phosphate (PI(4)P₁), and phosphatidylinositol-5-phosphate (PI(5)P₁).

5. A method for treating an individual having cancer, comprising administering to said individual a composition comprising a cSrc inhibitor, wherein inhibiting cSrc abates cancer progression.

6. The method of claim 5, wherein said cSrc inhibitor is a pharmaceutical composition of claim 3.

7. The method of claim 6, wherein said cSrc inhibitor is selected from the group consisting of phosphatidic acid, phosphatidylinositol-3-phosphate (PI(3)P₁), phosphatidylinositol-4-phosphate (PI(4)P₁), and phosphatidylinositol-5-phosphate (PI(5)P₁).

8. A method for treating an individual exhibiting resistance to chemotherapy, comprising administering to said individual a composition comprising a cSrc inhibitor, wherein inhibiting cSrc decreases resistance to chemotherapy.

9. The method of claim 8, wherein said cSrc inhibitor is a pharmaceutical composition of claim 3.

10. The method of claim 9, wherein said cSrc inhibitor is selected from the group consisting of phosphatidic acid, phosphatidylinositol-3-phosphate (PI(3)P₁), phosphatidylinositol-4-phosphate (PI(4)P₁), and phosphatidylinositol-5-phosphate (PI(5)P₁).

11. A method for treating an individual having cancer, comprising administering to said individual a composition that binds to the PH1 domain of human AFAP.

12. The method of claim 11, wherein said composition is a pharmaceutical composition of claim 3.

13. The method of claim 12, wherein said composition is selected from the group consisting of phosphatidic acid, phosphatidylinositol-3-phosphate (PI(3)P₁), phosphatidylinositol-4-phosphate (PI(4)P₁), and phosphatidylinositol-5-phosphate (PI(5)P₁).

14. A method for inhibiting cSrc activation, comprising treating cells with a composition comprising a compound selected from the group consisting of compounds of Formula (II): 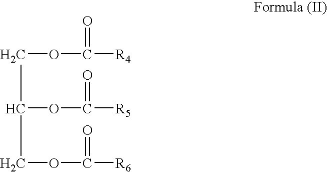
wherein R₄ and R₅ are selected independently and represent a linear or branched alkyl group containing 4 to 30 carbon atoms, a linear or branched alkenyl group containing 4 to 30 carbon atoms, or a linear or branched alkynyl group containing 4 to 30 carbon atoms, wherein these groups may comprise a cycloalkane ring or an aromatic ring;
wherein R₆ is selected from hydrogen, deuterium, tritium, phosphatidylinositol, phosphatidylinositol-4 phosphate, phosphatidylinositol-5-phosphate, phosphatidylinositol 3-phosphate, a linear or branched alkyl group containing 1 to 4 carbon atoms, a linear or branched alkenyl group containing 2 to 4 carbon atoms, and a linear or branched alkynyl group containing 2 to 4 carbon atoms, chlorine, bromine, fluorine, or iodine;
its pharmaceutically acceptable esters, and its pharmaceutically acceptable salts;
wherein said compound inhibits cSrc activation.

15. A pharmaceutical composition comprising a cSrc inhibitor of the general formula of Formula (II), below: 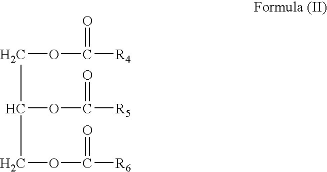
wherein R₄ and R₅ are selected independently and represent a linear or branched alkyl group containing 4 to 30 carbon atoms, a linear or branched alkenyl group containing 4 to 30 carbon atoms, or a linear or branched alkynyl group containing 4 to 30 carbon atoms, wherein these groups may comprise a cycloalkane ring or an aromatic ring;
wherein R₆ is selected from hydrogen, deuterium, tritium, phosphatidylinositol, phosphatidylinositol-4 phosphate, phosphatidylinositol-5-phosphate, phosphatidylinositol 3-phosphate, a linear or branched alkyl group containing 1 to 4 carbon atoms, a linear or branched alkenyl group containing 2 to 4 carbon atoms, and a linear or branched alkynyl group containing 2 to 4 carbon atoms, chlorine, bromine, fluorine, or iodine.

16. A method for treating an individual having cancer, comprising administering to said individual an effective amount of a pharmaceutical composition comprising a composition of claim 15.

17. A method for treating an individual exhibiting resistance to chemotherapy, comprising administering to said individual a composition comprising a composition of claim 15.

18. A method for treating an individual having cancer, comprising administering to said individual a composition of claim 15.
